(19) United States
(12) Patent Application Publication
(10) Pub. No.: US 2010/0121091 A1
(43) Pub. Date: May 13, 2010

(54) Processes of preparing asymmetric dinitrobenzamide mustard compounds, intermediate compounds useful therein and products obtained therefrom

(75) Inventors: Graham John Atwell, Auckland (NZ); William Alexander Denny, Auckland (NZ); Shangjin Yang, Auckland (NZ)

Correspondence Address: NIXON & VANDERHYE, PC, 901 NORTH GLEBE ROAD, 11TH FLOOR, ARLINGTON, VA, 22203, US

(73) Assignee: AUCKLAND UNISERVICES LIMITED, Auckland (NZ)

(21) Appl. No.: 12/310,484

(22) PCT Filed: Sep. 4, 2007
(86) PCT No.: PCT/NZ2007/000253

§ 371 Date: Nov. 19, 2009

(30) Foreign Application Priority Data
Sep. 4, 2006 (NZ) 549659

(51) Int. Cl. C07C 303/28; C07F 9/09

(52) U.S. Cl. 558/45; 558/48

(57) ABSTRACT

The invention relates to methods of preparing compounds of formula (II) wherein Z represents —OR¹ or N(R²)R^{2a}—, where R¹ is lower alkylene (C₁-C₆), R² is lower alkyl or H and R^{2a} is lower alkylene (C₁-C₆) or H; Q is absent when R^{2a} is H and is otherwise selected from the group consisting of H, —OH and protected forms of —OH; one of X and Y is halogen and the other is —OSO₂R³, where R³ is selected from the group consisting of lower alkyl (C₁-C₆), phenyl and CH₂phenyl. The method comprises the steps of: (a) reacting a compound of formula (I) with aziridineethanol or a 2-[(2-haloethyl)amino]ethanol in the presence of a metal halide, to form a compound of the formula (III) wherein one of X and E is halogen and the other is hydroxy, and (b) reacting the compound of formula (III) with an alkyl- or arylsulfonyl halide or alkyl- or arylsulfonyl anhydride to obtain a compound of the formula (II). The invention also relates to methods of preparing compounds of formula (IV) from the compounds of formula (II) so obtained, and to novel compounds of formula (IIb) useful as intermediates in these methods.

### FIELD OF THE INVENTION

The present invention relates generally to processes of preparing asymmetric mustards, particularly halomesylate mustards and especially 2-halomesylate-3,5-dinitrobenzamide mustards. The invention also relates to compounds useful as intermediates in such processes. The invention further relates to the use of compounds prepared by these processes as cytotoxins for cancer therapy and as bioreductive prodrugs for hypoxia, gene-directed enzyme-prodrug therapy (GDEPT), and antibody-directed enzyme-prodrug therapy (ADEPT).

### BACKGROUND TO THE INVENTION

Symmetric (hetero)aromatic mustards are well-known compounds widely used in cancer therapy, both as direct cytotoxins (e.g, 1; melphalan) (Feyns, Analytical Profiles of Drug Substances, 1984, 13 265), and as prodrugs for hypoxia (e.g., 2,3) (Palmer et al., J. Med. Chem., 1990, 33, 112; Lee et al., Bioorg. Med. Chem. Lett., 1998, 8, 1741), antibody-directed enzyme-prodrug therapy (ADEPT) (e.g., 4) (Springer et al, J. Med. Chem., 1995, 38, 5051), and gene-directed enzyme-prodrug therapy (GDEPT) (e.g., 5) (Niculescu-Duvaz et al., J. Med. Chem., 2003, 46, 1690).

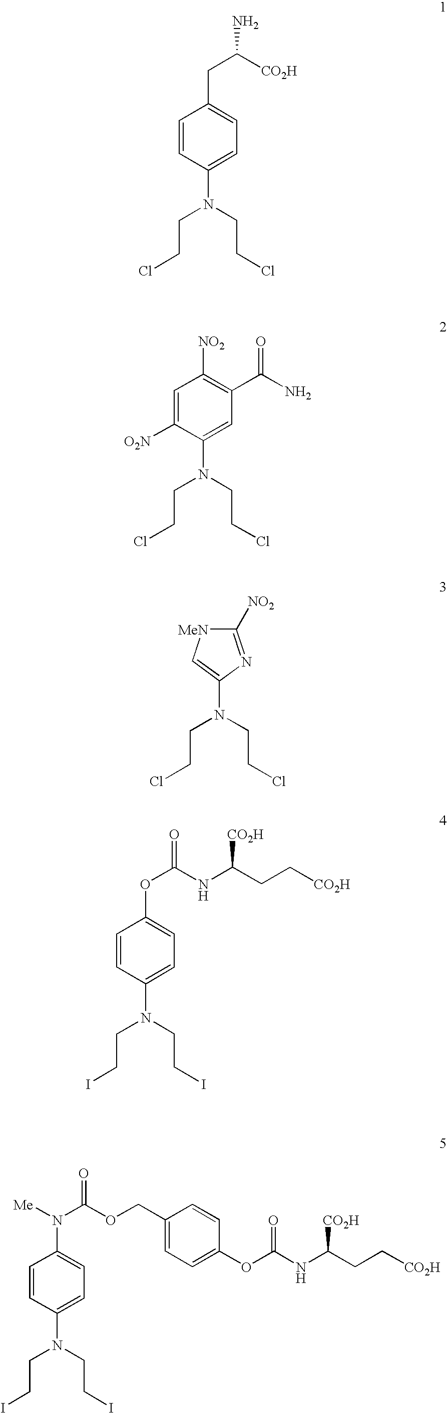

Asymmetric (hetero)aromatic mustards are less well-known, but have also been described for use as prodrugs for hypoxia (e.g., 6) [Denny et al., PCT Int. Appl. WO 04033415 A1], and (e.g., 7) in ADEPT [Pedley et al., Cancer Res., 1999, 59, 3998), and GDEPT (Springer et al., PCT Int. Appl. WO01085960 A1].

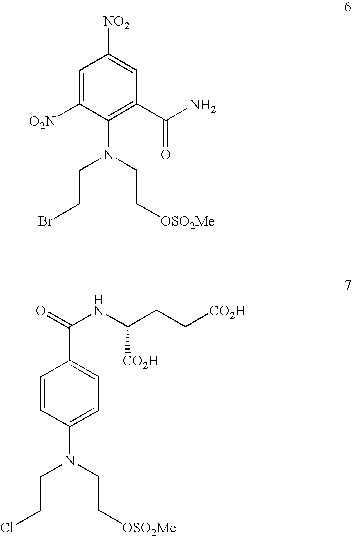

Several synthetic routes to asymmetric aromatic mustards are known:

Method 1. Stepwise alkylation of aromatic amines. (Scheme 1) [Mann et al., Tetrahedron, 1990, 46, 5377]. This method employs a three-step procedure; reductive alkylation with chlororacetaldehye/sodium cyanoborohydride, then oxirane to introduce the hydroxyethyl group, then mesylation, in an overall yield of less than 50%. Limitations of this method are the multi-step process and the moderate overall yield.

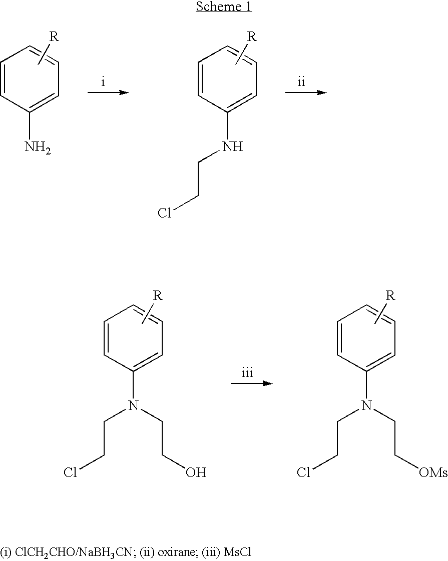

Method 2. Stoichiometric halogenation of bis(mesylates). (Scheme 2). [Denny et al., PCT Int. Appln. WO04033415 A1; ibid., PCT Int. Appnl. WO05042471 A1]. By reaction of dinitrobenzamide bis(mesylates) with a limited amount of alkyl halide. The major limitation of this method is the lack of selectivity for the bis(mesylate), which results in a mixture of all three components; starting material, asymmetric mustard, and the bis(mustard). The ratios of these can be influenced by the amount of halide used, to ensure the easiest separation, but careful chromatography is generally required, and yields of the desired asymmetric mustard are at best only about 40%.

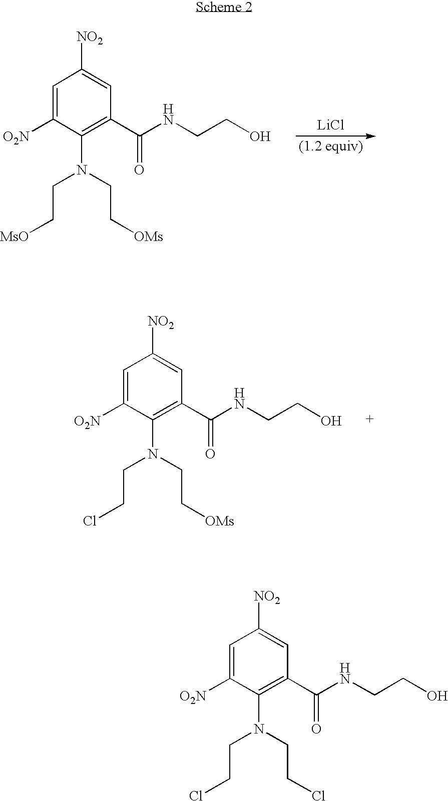

Method 3. Stoichiometric mesylation of bis(mustards). (Scheme 3). [Denny et al., PCT Int. Appnl. WO05042471 A1]. By reaction of (hetero)aromatic amine bis(mustards) with a limited amount of silver mesylate. This is analogous to Method 2, and the limitations are the same. A mixture of all three components; starting material, asymmetric mustard, and the bis(mustard) is generally obtained. Ratios can be influenced by the amount of silver mesylate used, to ensure the easiest separation, but careful chromatography is generally required, and yields of the desired asymmetric mustard are again usually only 30-40%.

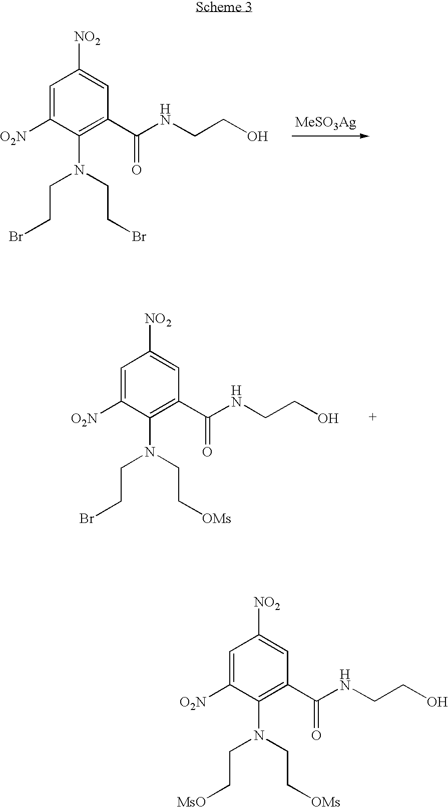

It is therefore an object of the invention to provide a synthetic method that is more convenient, and provides better yields of asymmetric dinitrobenzamide mustards than the existing ones, or to at least provide the public with a useful alternative.

### SUMMARY OF THE INVENTION

In a first aspect, the present invention provides a method of preparing a compound of formula (II)

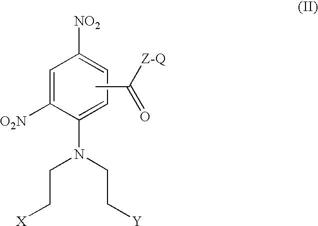

wherein:
Z represents —OR¹ or —N(R²)R^{2a}—,
where R¹ is lower alkylene (C₁-C₆), R² is lower alkyl or H and R^{2a} is lower alkylene (C₁-C₆) or H;
Q is absent when R^{2a} is H and is otherwise selected from the group consisting of H, —OH and protected forms of —OH;
one of X and Y is halogen and the other is —OSO₂R³,
where R³ is selected from the group consisting of lower alkyl (C₁-C₆), phenyl and CH₂phenyl;
the method comprising the steps of:
(a) reacting a compound of formula (I)

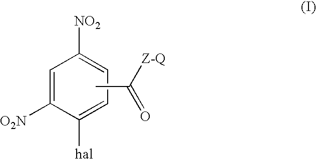

wherein hal is a halogen atom, Z is as defined above for formula (II) and Q is absent when R^{2a} is H and is otherwise selected from the group consisting of H and protected forms of OH,
with aziridineethanol or a 2-[(2-haloethyl)amino]ethanol in the presence of a metal halide, to form a compound of the formula (III):

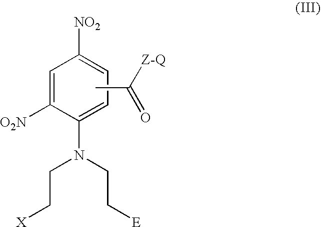

wherein one of X and E is halogen and the other is hydroxy, and Z and Q are as defined above, and
(b) reacting the compound of formula (III) so obtained with an alkyl- or arylsulfonyl halide or alkyl- or arylsulfonyl anhydride to obtain a compound of the formula (II).

In another aspect, the invention provides a method of preparing a compound of formula (III) as defined above, the method comprising the step of reacting a compound of formula (I) as defined above with aziridineethanol or a 2-[(2-haloethyl)amino]ethanol in the presence of a metal halide.

In a further aspect, the invention provides a method of preparing a compound of the formula (IV)

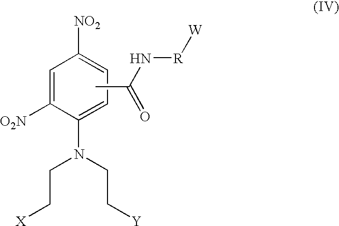

where W is —OH or —OP(O)(OH)₂;
R is lower alkylene (C₁-C₆);
and one of X and Y is halogen and the other is —OSO₂R³; wherein R³ is selected from the group consisting of lower alkyl (C₁-C₆), phenyl and CH₂phenyl;
the method comprising the steps of:
(a) reacting a compound of formula (I) as defined above with aziridineethanol or a 2-[(2-haloethyl)amino]ethanol in the presence of a metal halide to obtain a compound of formula (III) as defined above;
(b) reacting the compound of formula (III) so obtained with an alkyl- or arylsulfonyl halide or alkyl- or arylsulfonyl anhydride to obtain a compound of formula (II) as defined above; and
(c) optionally, carrying out further processing of the compound of formula (II) as required to obtain a compound of formula (IV).

In still a further aspect, the invention provides a process of preparing a compound of formula (IV) as defined above, characterised in that the process includes the step of reacting a compound of formula (I) as defined above with aziridineethanol or a 2-[(2-haloethyl)amino]ethanol in the presence of a metal halide to obtain a compound of formula (III) as defined above.

In still a further aspect, the invention provides a process of preparing a compound of formula (II) as defined above, characterised in that the process includes the step of reacting a compound of formula (I) as defined above with aziridineethanol or a 2-[(2-haloethyl)amino]ethanol in the presence of a metal halide to obtain a compound of formula (III) as defined above.

In a further aspect, the invention provides a compound of formula (IIb)

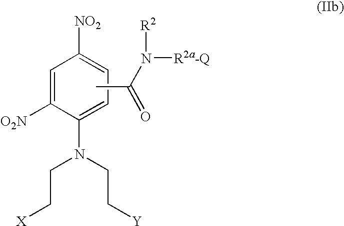

wherein:
one of X and Y is halogen and the other is —OSO²R³, where R³ is selected from the group consisting of lower alkyl (C₁-C₆), phenyl and CH₂phenyl;
R² represents lower alkyl (C₁-C₆), or H, and R^{2a} represents lower alkylene (C₁-C₆);
Q is selected from the group consisting of:
(1) —OR⁴, where R⁴ is a mono-, di- or tripeptide,
(2) —OR⁶, where R⁶ is a mono-, di- or trisaccharide,
(3) —O(C═O)K, where K is (a) lower alkyl (C₁-C₆) optionally substituted with one or more groups selected from OH, NH₂, NHR⁵ and NR⁵R^{5a}, where each R⁵ and R^{5a} is independently lower alkyl (C₁-C₃), or R⁵R^{5a} taken together represents pyrrolyl, piperidinyl, piperazinyl, 4-methylpiperazinyl, N-imidazolyl or 2-, 3- or 4-pyridyl, or K is (b) (CH₂)ₙCONH(CH₂)ₙNR⁵R^{5a}, where n is 1, 2 or 3, and R⁵ and R^{5a} are as defined above, and

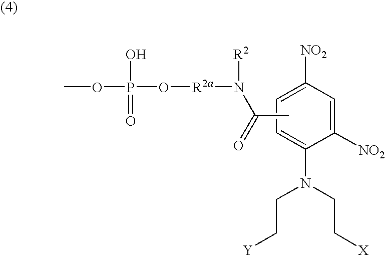

wherein R², R^{2a}, X and Y are as defined immediately above;
and wherein, when R² is lower (C₁-C₆) alkyl, Q can also represent —OP(O)(OH)₂.

Preferred compounds of formula (IIb) include compounds of formula (V):

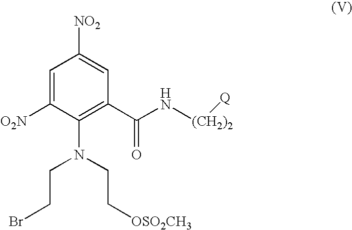

wherein
Q is selected from the group consisting of:
(1) —OR⁴, where R⁴ is a mono-, di- or tripeptide,
(2) —OR⁶, where R⁶ is a mono-, di- or trisaccharide,
(3) —O(C═O)K, where K is (a) lower alkyl (C₁-C₆) optionally substituted with one or more groups selected from OH, NH₂, NHR⁵ and NR⁵R^{5a}, where each R⁵ and R^{5a} is independently lower alkyl (C₁-C₃), or R⁵R^{5a} taken together represents pyrrolyl, piperidinyl, piperazinyl, 4-methylpiperazinyl, N-imidazolyl or 2-, 3- or 4-pyridyl, or K is (b) (CH₂)ₙCONH(CH₂)ₙNR⁵R^{5a}, where n is 1, 2 or 3, and R⁵ and R^{5a} are as defined above, and

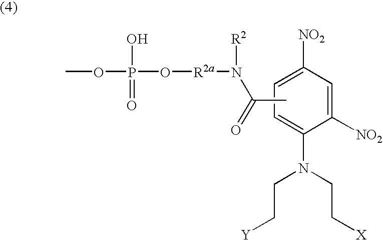

wherein R², R^{2a}, X and Y are as defined immediately above.

A preferred subset of compounds of formula (II) to be prepared from a precursor halide of formula (I) are those where a halide of formula (IIa) is prepared from a precursor halide of formula (Ia),

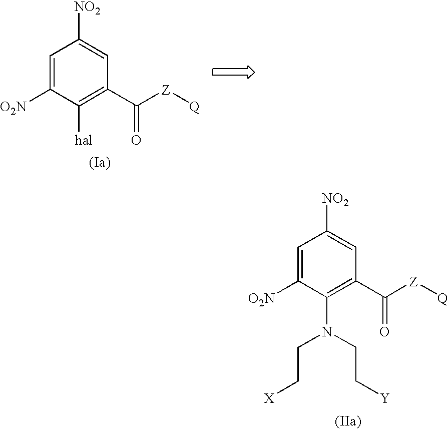

wherein:
Z-Q in formulae (Ia) and (IIa) represents OtBu or NH(CH₂)ₙOJ, where n is 2 or 3 and J is THP (tetrahydropyranyl), P(O)(OtBu)₂, methyl tetraacetyl-β-glucuronide or COK, where K is a protected mono-, di- or tripeptide;
hal in formula (Ia) represents Cl, Br or I; and
one of X and Y in formula (IIa) represents halogen and the other represents —OSO₂R³, where R³ is lower alkyl (C1-C6).

Another preferred subset of compounds of formula (IIa) to be prepared from a precursor halide of formula (Ia) is where:

Z-Q in formulae (Ia) and (IIa) represents OtBu or NH(CH₂)ₙOJ, where n is 2 or 3 and J is THP (tetrahydropyranyl), P(O)(OtBu)₂, or methyl tetraacetyl-β-glucuronide;
hal in formula (Ia) represents Cl or Br, and
one of X and Y in formula (IIa) represents halogen and the other represents —OSO₂Me.

A further preferred subset of compounds of formula (IIa) to be prepared from a precursor halide of formula (Ia) is where;

Z-Q in formulae (Ia) and (IIa) represents OtBu or NH(CH₂)₂OJ, where J is THP (tetrahydropyranyl) or P(O)(OtBu)₂;
hal in formula (Ia) represents Cl or Br, and
X and Y in formula (IIa) separately represent Br and OSO₂Me.

In another aspect, the present invention provides specific compounds of general formula (II) obtained by the processes defined above. Preferably such compounds are selected from the following:
2-[5-(aminocarbonyl)(2-chloroethyl)-2,4-dinitroanilino]ethyl methanesulfonate;
2-[2-(aminocarbonyl)(2-chloroethyl)-4,6-dinitroanilino]ethyl methanesulfonate;
2-[2-(aminocarbonyl)(2-bromoethyl)-4,6-dinitroanilino]ethyl methanesulfonate;
2-[2-(aminocarbonyl)(2-iodoethyl)-4,6-dinitroanilino]ethyl methanesulfonate;
2-[(2-chloroethyl)-2,4-dinitro-6-({[2-(tetrahydro-2H-pyran-2-yloxy)ethyl]amino}carbonyl)-anilino]ethyl methanesulfonate;
2-[(2-bromoethyl)-2,4-dinitro-6-({[2-(tetrahydro-2H-pyran-2-yloxy)ethyl]amino}carbonyl)-anilino]ethyl methanesulfonate;
2-[(2-iodoethyl)-2,4-dinitro-6-({[2-(tetrahydro-2H-pyran-2-yloxy)ethyl]amino}carbonyl)-anilino]ethyl methanesulfonate;
*2-[(2-bromoethyl)-2,4-dinitro-6-({[2-(tetrahydro-2H-pyran-2-yloxy)ethyl]amino}carbonyl)-anilino]ethyl 1-butanesulfonate;
*2-[2-(6-tert-butoxy-8,8-dimethyl-6-oxido-5,7-dioxa-2-aza-6-phosphanon-1-anoyl)(2-chloroethyl)-4,6-dinitroanilino]ethyl methanesulfonate;
   or physiologically functional derivatives thereof.

The latter two compounds (marked with *) and their derivatives have novelty independent of the process by which they are made.

In another aspect, the present invention provides specific compounds of the formula (IV) obtained from the products (II) of the above defined transformation, by further processes outlined in Schemes 5-10, and described in Examples 1-20 below, or by similar processes, thus providing an overall improved synthesis of said compounds. A specially preferred set of such compounds are:
2-((2-chloroethyl)-2-{[(2-hydroxyethyl)amino]carbonyl}-4,6-dinitroanilino)ethyl methanesulfonate;
2-((2-bromoethyl)-2-{[(2-hydroxyethyl)amino]carbonyl}-4,6-dinitroanilino)ethyl methanesulfonate;
2-((2-iodoethyl)-2-{[(2-hydroxyethyl)amino]carbonyl}-4,6-dinitroanilino)ethyl methanesulfonate;
2-((2-bromoethyl)-2-{[(2-hydroxyethyl)amino]carbonyl}-4,6-dinitroanilino)ethyl 1-butanesulfonate;
2-[(2-bromoethyl)-2,4-dinitro-6-[[[2-(phosphonooxy)ethyl]amino]carbonyl]anilino]ethyl methanesulfonate;
2-[(2-bromoethyl)-2,4-dinitro-6-({[2-(phosphonooxy)ethyl]amino}carbonyl)anilino]ethyl 1-butanesulfonate;
2-[(2-chloroethyl)-2,4-dinitro-6-[[[2-(phosphonooxy)ethyl]amino]carbonyl]anilino]ethyl methanesulfonate;
2-[(2-iodoethyl)-2,4-dinitro-6-({[2-(phosphonooxy)ethyl]amino}carbonyl)-anilino]ethyl methanesulfonate.

A preferred compound of the formula IV obtained from the products (II) of the above defined transformation, by the further processes outlined in Scheme 7 and described in Example 9 below, or by similar processes, is 2-[(2-bromoethyl)-2,4-dinitro-6-[[[2-(phosphonooxy)ethyl]amino]-carbonyl]anilino]ethyl methanesulfonate (33b) [Denny et al., WO 05042471], or a physiologically functional derivative thereof.

In another aspect, the present invention relates to the use of the compounds obtained from one of the processes defined above as anticancer drugs. The compounds so obtained may be used for the selective killing of oxic and hypoxic tumour cells in methods of treatment of cancers, for example leukemias and particularly solid cancers including breast, bowel and lung tumours, including small cell lung carcinoma.

In a further aspect, the present invention further relates to the use of some of the compounds obtained by the processes defined above that are suitable as substrates for nitroreductase or carboxypeptidase enzymes (for example, the aerobic NR2 nitroreductase isolated from E. coli) in methods of ADEPT and GDEPT therapy.

It is recognised that certain compounds produced by the methods of the present invention may exist in one of two different enantiomeric or diastereomeric forms. In such cases it is to be understood that the compounds prepared by the processes of the invention may represent either enantiomeric or diastereomeric form or a mixture of both.

A halogen group or -hal depiction used throughout the specification is to be taken as meaning a fluoro, chloro, bromo or iodo group.

Physiologically functional derivatives of the compounds that are obtained by the processes defined above are to be understood as including salts, amides and esters. Esters include carboxylic acid esters in which the non-carbonyl moiety of the ester grouping is selected from straight or branched chain C₁₋₆ alkyl, (methyl, n-propyl, n-butyl or t-butyl); or C₃₋₆ cyclic alkyl (e.g. cyclohexyl), or a chain of from one to three D- or L-aminoacids. Salts include physiologically acceptable base salts, e.g. derived from an appropriate base, such as alkali metal (e.g. sodium), alkaline earth metal (e.g. magnesium) salts, ammonium and NR⁴″ (wherein R⁴″ is C₁₋₄ alkyl) salts. Other salts include acid addition salts, including the hydrochloride and acetate salts. Amides include non-substituted and mono- and di-substituted derivatives. Such derivatives may be prepared by techniques known per se in the art of pharmacy.

Further aspects of the present invention will become apparent from the following description given by way of example only, and with reference to the accompanying synthetic schemes.

### DETAILED DESCRIPTION OF THE INVENTION

The present invention relates to methods of preparing compounds of formula (II) as defined above. In turn, the compounds of formula (II) may be converted by further processing steps, as required, to obtain compounds of the formula (IV). Compounds of the formula (IV) and their use in anticancer therapy are described in WO 05/042471.

The synthetic methods of the present invention have been found, at least in preferred embodiments, to be more convenient and/or to provide greater yields of the compounds of formulae (II) and (IV) than methods previously known.

The process for preparing a compound of formula (II) includes the steps of reacting a halide of formula (I) with a 2-[(2-haloethyl)amino]ethanol, preferably 2-[(2-chloroethyl)amino]ethanol, or even more preferably with aziridineethanol, in the presence of a metal halide, such as a lithium halide. The reaction is preferably carried out in a dry solvent such as tetrahydrofuran, methyl ethyl ketone or methyl isopropyl ketone or, preferably, 1,4-dioxane (for example, as in Scheme 4). This is followed by reaction of the resulting haloalcohol of the formula (III) with an alkyl- or arylsulfonyl halide, such as methanesulfonyl chloride, or alkyl- or arylsulfonyl anhydride, such as methansulfonic anhydride, to obtain a compound of the formula (II).

The 2-[(2-haloethyl)amino]ethanols such as 2-[(2-chloroethyl)amino]ethanol can be prepared by known methods ([e.g., W. C. J. Ross and J. G. Wilson, J. Chem. Soc. 1959, 3616; I. Aiko et al, Yakugaku Zasshi, 1955, 75, 418].

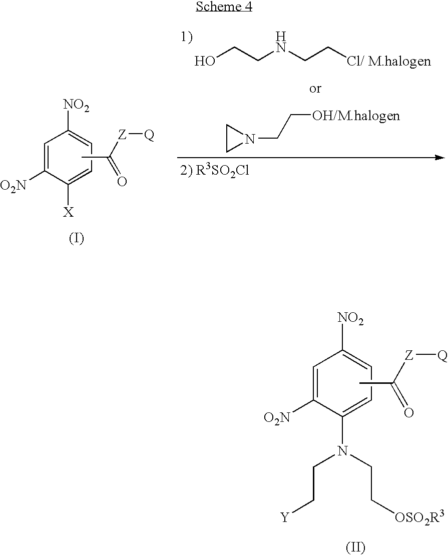

In Scheme 4, M represents a group I or group II metal ion.

As described above, the starting materials for the methods of the present invention are compounds of the formula (I):

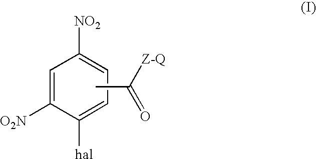

where hal is a halogen atom,
Z represents —OR¹— or —N(R²)R^{2a},
where R¹ is lower alkylene (C₁-C₆),
R² is lower alkyl or H and R^{2a} is lower alkylene (C₁-C₆) or H; and
Q is absent when R^{2a} is H and is otherwise selected from the group consisting of: H and protected forms of —OH.

As indicated above, the group Q may be H or a protected form of —OH. Suitable protecting groups for —OH are known in the art. Protected forms of —OH include, but are not limited to, ethers, phosphate esters, methyl tetraacetyl glucuronates, peracetyl glycosides and amino acid polypeptide esters.

In certain preferred embodiments, Q is selected from the group consisting of

(1) —OR⁴, where R⁴ is a mono-, di- or tripeptide,
(2) —OR⁶, where R⁶ is a mono-, di- or trisaccharide,
(3) —O(C═O)K, where K is (a) lower alkyl (C₁-C₆) optionally substituted with one or more groups selected from OH, NH₂, NHR⁵ and NR⁵R^{5a}, where each R⁵ and R^{5a} is independently lower alkyl (C₁-C₃), or R⁵R^{5a} taken together represents pyrrolyl, piperidinyl, piperazinyl, 4-methylpiperazinyl, N-imidazolyl or 2-, 3- or 4-pyridyl, or K is (b) (CH₂)ₙCONH(CH₂)ₙNR⁵R^{5a}, where n is 1, 2 or 3, and R⁵ and R^{5a} are as defined above, and

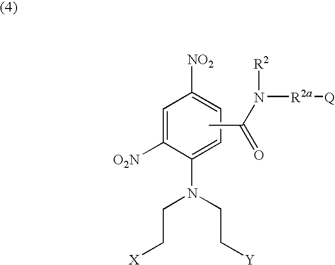

wherein R², R^{2a}, X and Y are as defined immediately above, and

### (5) —OP(O)(OH)₂.

Compounds of the formula (I) are known compounds and can be prepared using methods known in the art, such as those described in WO 05/042471. For example, the following general method, as shown in Scheme 4a below, may be used. Acids (Ia) can be converted directly to esters (I) by reaction with alcohols in the presence of acid, or by reaction with tert-butyl acetate in the presence of acid. Acids (Ia) can also be activated by reaction with reagents such as thionyl chloride or bromide, phosphorous oxychloride or oxybromide, or preferably oxalyl chloride or oxalyl bromide, or similar reagents, to give acid halides (Ia) where A is Cl or Br. These acid halides can then be reacted directly with alcohols to give esters (I). They can also be reacted with amines, either directly or preferably in an inert moderating solvent, with the optional presence of acid scavengers such as triethylamine, to give amides (I). Acids (Ia) can also be reacted with coupling reagents such as CDI, DECP or EDCI to give activated intermediates (Ic), which can be reacted with alcohols or amines as above to give esters (I) or amides (I).

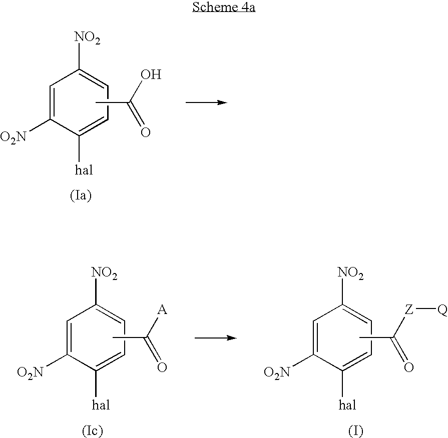

The compounds of formula (II) may then be converted to desired compounds of formula (IV) using further processing steps that are also well known in the art. For example, compounds of formula (II) where Z=—OR¹ and Q=H can be deblocked by treatment with acids or alkalis to give acids (e.g., 37), which can be activated and reacted with suitable amines to give compounds of formula (IV). Compounds of formula (II) where Z=—OR¹ and Q=protected OH can be deblocked by treatment with acids or alkalis, followed if desired by reaction with phosphorylating agents such as di-tert-butyl diethylphosphoramidite and 1H-tetrazole or 1,5-dicyanoimidazole, followed by mild oxidation and phosphate ester hydrolysis with mild acid such as trifluoroacetic acid to give compounds of formula (IV) where Z=—OR¹ and Q=OP(O)(OH)₂.

As indicated above, one preferred compound of formula (IV) that can be prepared by the methods of the present invention is 2-[(2-bromoethyl)-2,4-dinitro-6-[[[2-(phosphonooxy)ethyl]amino]carbonyl]anilino]ethyl methanesulfonate (33b). In certain preferred embodiments, this compound is prepared by the methods shown in Reaction Scheme 16 and described below.

Accordingly, in certain preferred embodiments, the invention provides a method of preparing a compound of the formula (IId) (which is within the general formula (II) defined earlier):

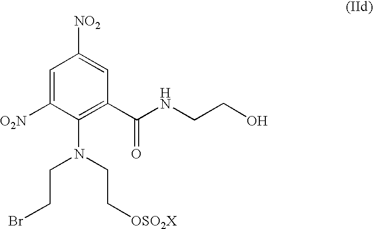

wherein X is alkyl or aryl;
wherein the method comprises the following steps:
(a) reacting a compound of the general formula (I) as defined earlier having the following formula:

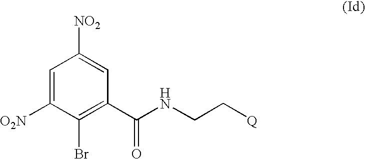

wherein Q is a protected form of —OH, with aziridineethanol or a 2-[(2-haloethyl)amino]ethanol in the presence of a metal halide, to form a compound of the general formula (III) as defined earlier having the following formula:

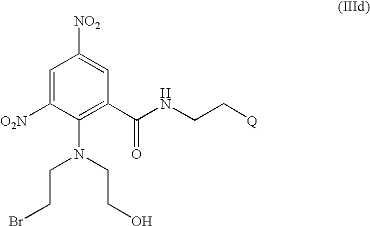

wherein Q is as defined immediately above; and
(b) reacting the compound of formula (IIId) with an alkyl- or arylsulfonyl halide or alkyl- or arylsulfonyl anhydride to form a compound of the Formula (IId) as defined above.

In certain preferred embodiments of the above method, the compound of formula (Id) is reacted with aziridine ethanol, in the presence of a metal halide, such as sodium bromide.

It is also preferred that the alkyl- or arylsulfonyl halide or anhydride used in step (b) is to methansulfonic anhydride, and that the compound of formula (IId) prepared is of the formula:

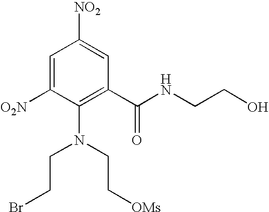

In the above method, the compound of formula (Id) may conveniently be prepared by reacting a compound of the formula:

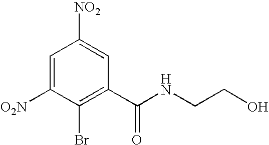

with dihydropyran, to form a compound of formula (Id) as defined above, in which Q is THP.

In preferred embodiments, the above method includes the additional step of further processing the compound of formula (IId) as defined above to form a compound of the following formula:

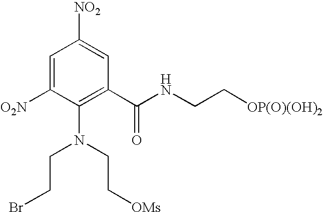

In certain preferred embodiments, the further processing comprises reacting the compound of formula (IId) with iPr₂NP(OtBu), to form a phosphate ester of the compound of formula (IId), followed by deprotecting the phosphate ester. The phosphate ester may conveniently be deprotected using MsOH.

Certain compounds of formula (II) are novel. Accordingly, the present invention provides as a further feature compounds of the formula (IIb):

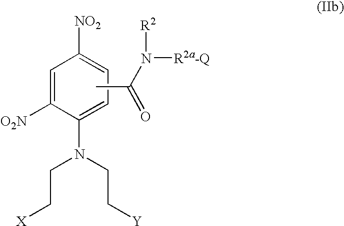

wherein:
one of X and Y is halogen and the other is —OSO²R³, where R³ is selected from the group consisting of lower alkyl (C₁-C₆), phenyl and CH₂phenyl;
R² represents lower alkyl (C₁-C₆), or H, and R^{2a} represents lower alkylene (C₁-C₆);
Q is selected from the group consisting of:
(1) —OR⁴, where R⁴ is a mono-, di- or tripeptide,
(2) —OR⁶, where R⁶ is a mono-, di- or trisaccharide,
(3) —O(C═O)K, where K is (a) lower alkyl (C₁-C₆) optionally substituted with one or more groups selected from OH, NH₂, NHR⁵ and NR⁵R^{5a}, where each R⁵ and R^{5a} is independently lower alkyl (C₁-C₃), or R⁵R^{5a} taken together represents pyrrolyl, piperidinyl, piperazinyl, 4-methylpiperazinyl, N-imidazolyl or 2-, 3- or 4-pyridyl, or K is (b) (CH₂)ₙCONH(CH₂)ₙNR⁵R^{5a}, where n is 1, 2 or 3, and R⁵ and R^{5a} are as defined above, and

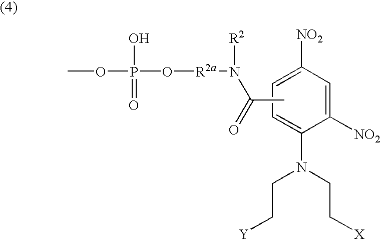

wherein R², R^{2a}, X and Y are as defined immediately above;
and wherein, when R² is lower (C₁-C₆) alkyl, Q can also represent —OP(O)(OH)₂.

The invention also provides the use of the compounds of formula (IIb) as intermediate compounds to prepare compounds of the formula (IV).

At least in certain preferred embodiments, the method of the present invention is superior to the previous reported methods (Methods 1-3 described above), as shown by the comparative yields given in Table 1, and the ease of purification of the products. The compounds of Table 1 can be prepared by the general methods set out in Schemes 5-12 and exemplified in Examples 1-20 below.

Schemes 13 to 15 illustrate methods for preparing certain compounds of formula (IIb), which can in turn be converted to desired compounds of formula (IV) by the methods described above.

TABLE 1
Preparation of halomesylate mustards (II) from dinitrobenzamide halides (I).

| | | | | | Yield | | |
|---|---|---|---|---|---|---|---|
| | | | | | (steps) | Pre- | Ref (previous |
| Entry | I | | II | | (I → II) | vious | method) |
| 1 | 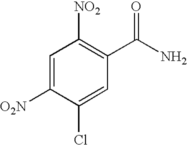 | 8 | 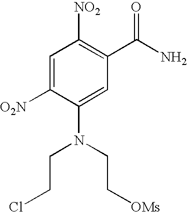 | 10a: X = Cl 10b: X = Br 10c: X = I | 42% (2) 37% (3) 29% (3) | 12% (3) new new | J. Med. Chem., 1992, 35, 3214; WO04033415 |
| 2 | 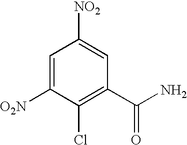 | 11 | 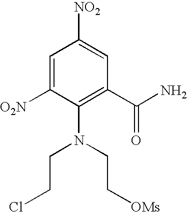 | 13a: X = Cl 13b: X = Br 13c: X = I | 92% (2) 74% (2) 45% (3) | 15% (3) 24% (3) | J. Med. Chem, 1997, 40, 1270; WO04033415 J. Med. Chem, 1997, 40, 1270; WO04033415 new |
| 3 | 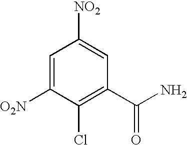 | 11 | 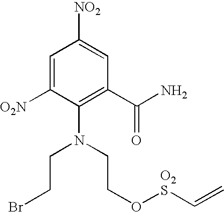 | 14 | 81% (2) | new | |
| 4 | 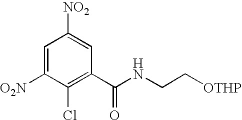 | | 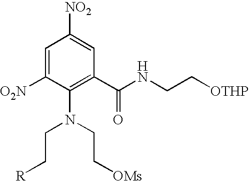 | 17a: R = Cl 17b: R = Br 17c: R = I | 95% (2) or 37% (2)^{c} 86% (2) 81% (2) | 40% (2) 40% (2) 19% (2) | WO04033415 WO04033415 WO2005042471 |
| 5 | 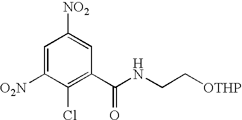 | 15 | 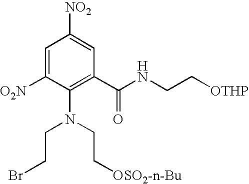 | 18 | 90% (2) | new | |
| 6 | 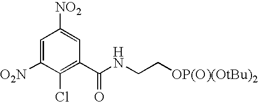 | 19 | 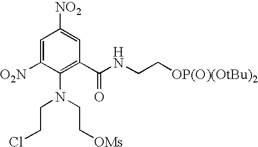 | 21 | 68% (2)^{c} | new | |
| 7 | 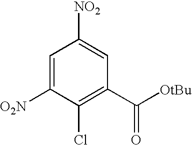 | 22 | 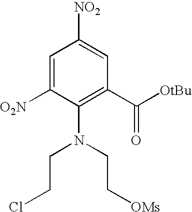 | 24 | 68% (2)^{c} | new | |
| 8 | 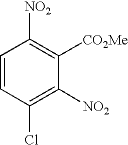 | 25 | 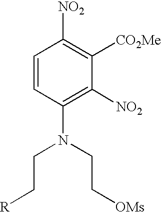 | 27a: R = Cl 27b: R = Br 27c: R = I | 20% (2) 28% (2) 53% (2) | new new new | |
| 9 | 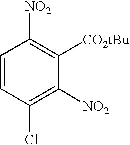 | 28 | 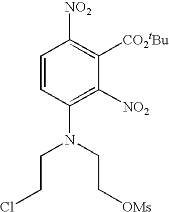 | 30 | 45% (2) | new | |

Footnotes for Table 1.
^{a}The method claimed in this application;
^{b}Previous methods (see above);
^{c}Using 2-[(2-chloroethyl)amino]ethanol as reagent for the first step.

With reference to the following Examples and Schemes, a synthetic route to the compounds of formula (II) is outlined, as defined in the first aspect of the invention above.

In Scheme 5, reaction of (for example) 2,4-dinitro-5-chlorobenzamide (8) with aziridineethanol in a suitable dry solvent, but preferably DMF, in the presence of an alkali metal chloride, but preferably lithium chloride, gave good yields of the corresponding chloroalcohols (e.g., 9a). These could then be converted to the corresponding chloromesylate mustards (e.g., 10a) with mesylating agents, preferably methanesulfonyl chloride. Other halomesylates (10b, 10c) can be prepared from 10a by reaction of it with the appropriate metal halides (preferable lithium bromide or lithium iodide), followed by reaction with mesylating agents, preferably methanesulfonyl chloride.

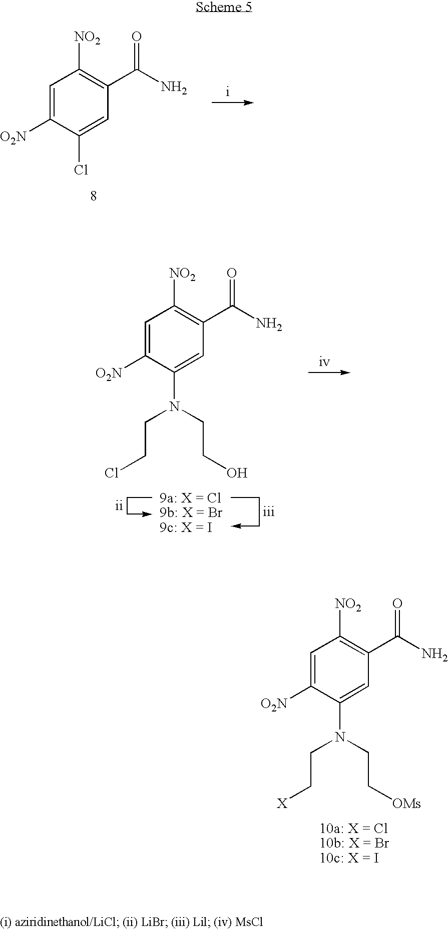

In Scheme 6, similar reaction of (for example) the 3,5-dinitro-2-chlorobenzamide (11) with aziridineethanol in a suitable dry solvent, but preferably DMF, in the presence of an alkali metal halides, preferably lithium halides, gave the haloalcohols (e.g., 12a-12c). These could then be converted to the corresponding haloalkylsulfonate mustards (e.g., 13a-13c, 14) with alkylsulfonating agents, preferably alkanesulfonyl chlorides.

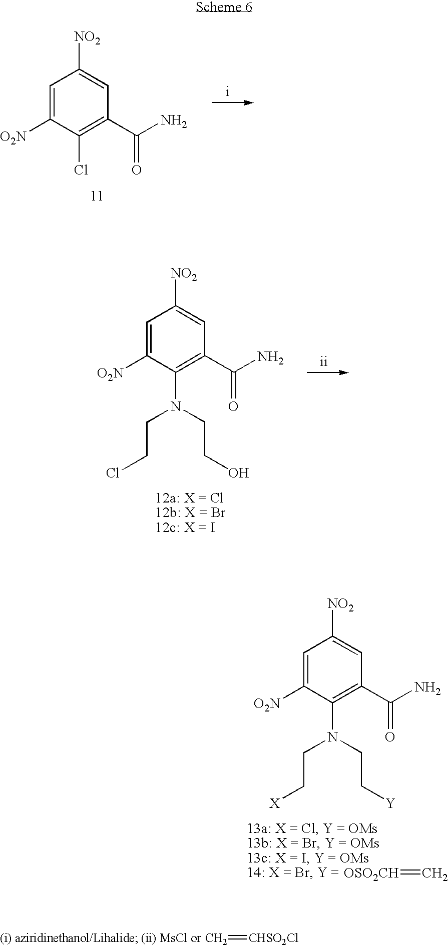

In Scheme 7, similar reaction of (for example) the protected THP ether 15 with aziridineethanol in a suitable dry solvent, but preferably DMF, in the presence of an alkali metal halide, but preferably a lithium halide, gave the corresponding haloalcohols (e.g., 16a-16c). These could then be converted to the corresponding halomesylate mustards (e.g., 17a-17c) with mesylating agents, preferably methanesulfonyl chloride. Deblocking of these with acid, preferably methansulfonic acid, gave the alcohols (31a-31c), which could be phosphorylated, preferably with di-tert-butyl diisopropylphosphoramidite, and then oxidised, preferably with hydrogen peroxide, to give the phosphate esters (32a-32c). Hydrolysis of these with acid, preferably trifluoroacetic acid, gave the phosphates (33a-33c).

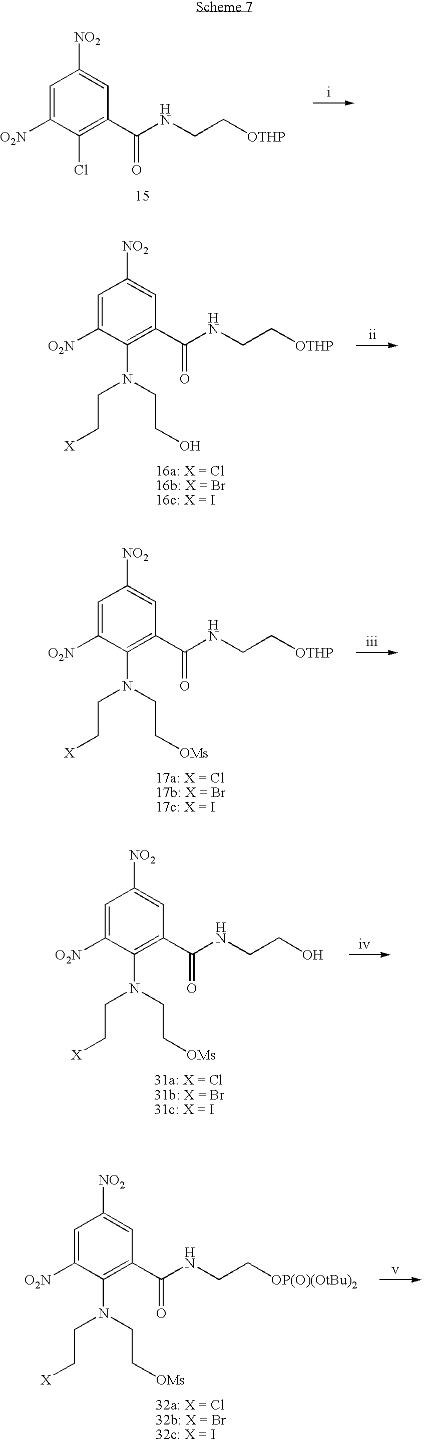

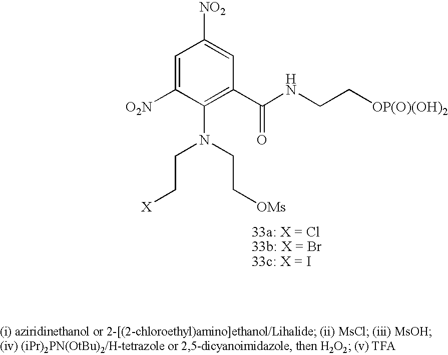

In Scheme 8, similar reaction of (for example) 16b with (for example) butanesulfonyl chloride gave bromoalkylsulfonate mustards (e.g., 18). Deblocking of this with acid, preferably methanesulfonic acid, gave the alcohol (34), which could be phosphorylated, preferably with di-tert-butyl diisopropylphosphoramidite, and then oxidised, preferably with hydrogen peroxide, to give the phosphate ester (35). Hydrolysis of this with acid, preferably trifluoroacetic acid, gave the phosphate (36).

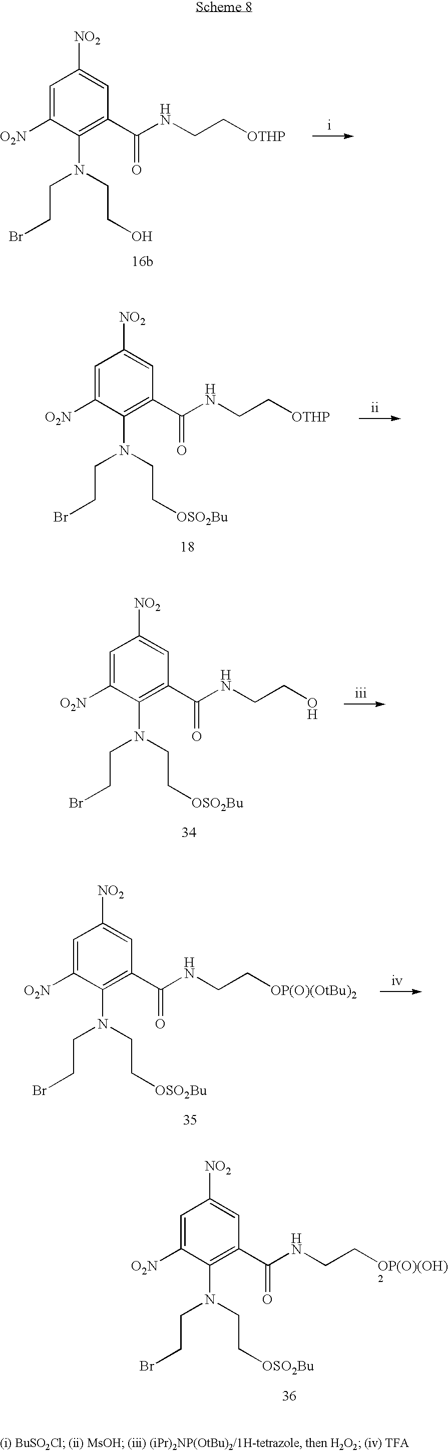

In Scheme 9, the phosphate ester 19 can be prepared either by reaction of the amide 37 with di-tert-butyl diisopropylphosphoramidite, or directly from reaction of the acid chloride 38 with 2-aminoethyl di-tert-butyl phosphate. Reaction of 19 with either aziridineethanol or 2-[(2-chloroethyl)amino]ethanol gave the chloroalcohol 20. Addition of metal halides (preferably lithium bromide or iodide) to the reaction, preferably with aziridineethanol, allows the similar preparation of bromo- and iodoalcohols. These can then be converted to the corresponding halomesylate mustard (e.g., chloromesylate 21) with mesylating agents, preferably methanesulfonyl chloride.

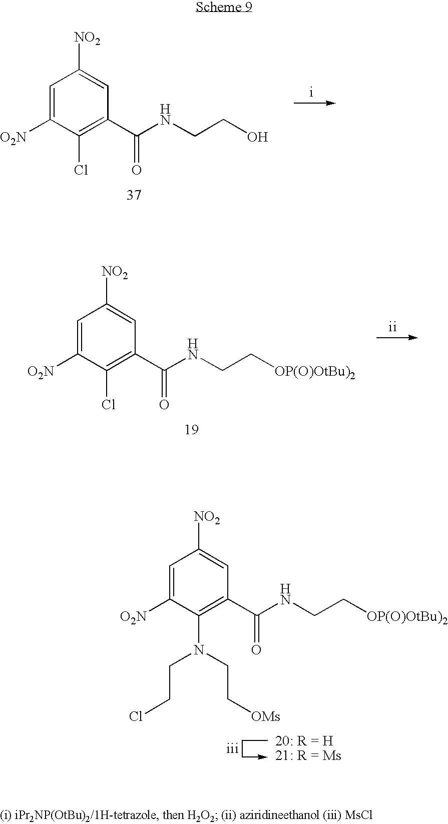

In Scheme 10, reaction of the known [Guenin & Schneider, Helv. Chim. Acta, 1983, 66, 1101] t-butyl ester 22 is reacted with aziridineethanol to give the chloroalcohol 23, which is mesylated as usual to give the chloromesylate 24. Hydrolysis with TFA to the acid 39, followed by activation with oxalyl chloride and coupling with 2-aminoethanol then gave amide 31a directly.

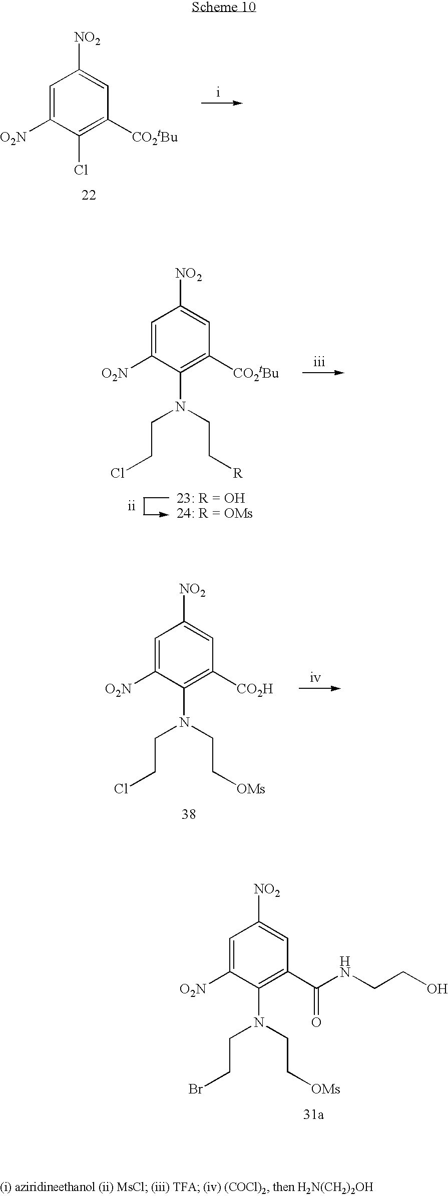

In Scheme 11, nitration of commercially-available 3-chloro-2-nitrobenzoic acid (40) with fuming nitric acid/conc. sulfuric acid is a new, high-yielding route to 3-chloro-2,6-dinitrobenzoic acid (41), which can be readily converted to the methyl ester (25). Reaction of this with aziridineethanol in the presence of the appropriate metal halides, preferably lithium halides, gives the haloalcohols (26a-26c), which can be converted to the halomesylates (27a-27c) in good yield.

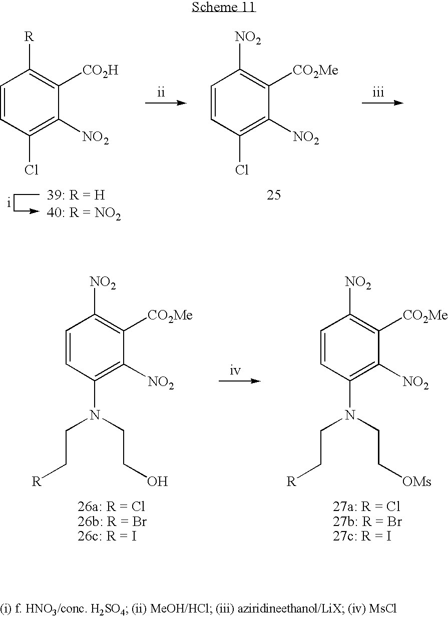

In Scheme 12, reaction of acid 40 with tert-butyl acetate and perchloric acid gives the tert-butyl ester (28) in good yield, and this is reacted with aziridineethanol in the presence of metal halides (example shown for lithium chloride) to give haloalcohols (example shown is the chloroalcohol 29), which can be converted to halomesylates (example shown is the chloromesylate 30) in good yield.

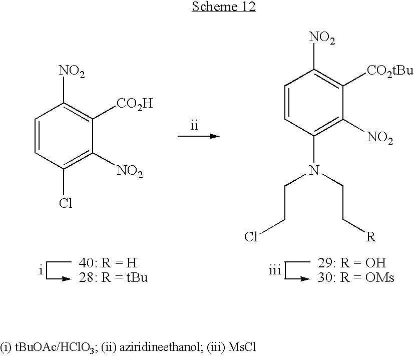

In Scheme 13, reaction of alcohol 37 with protected N—BOC valine (as example) followed by reaction of the subsequent compound 41 with aziridineethanol or a 2-[(2-haloethyl)amino]ethanol in the presence of a metal halide gives compound 42, which can be mesylated and deblocked to give 44. Alternatively, 37 can be OH-protected (e.g., THP ether, to give 15), and this can then be reacted as in Scheme 13.

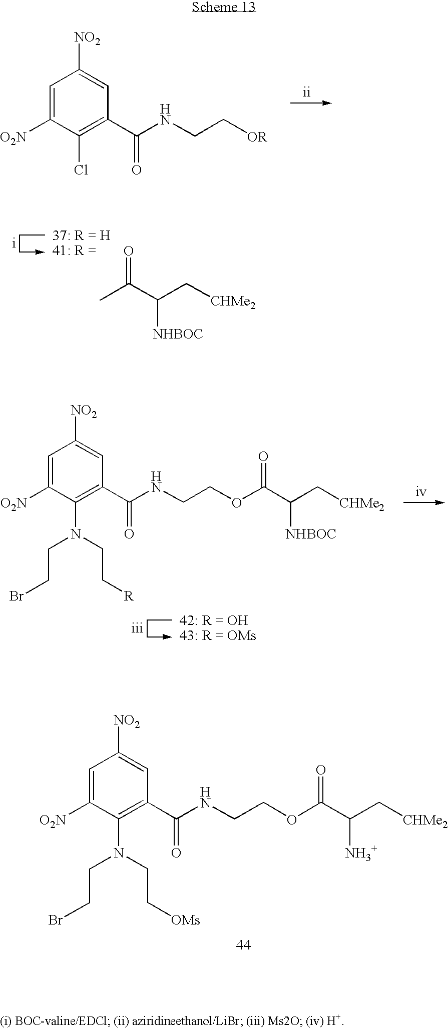

In Scheme 14, reaction of 37 with bromotetraacetylgalactose (as example), followed by reaction of the subsequent compound 45 with aziridineethanol or a 2-[(2-haloethyl)amino]ethanol in the presence of a metal halide gives compound 46, which can be mesylated and deblocked to give 48. Alternatively, 37 can be OH-protected (e.g., THP ether, to give 15), and this can be reacted as in Scheme 14.

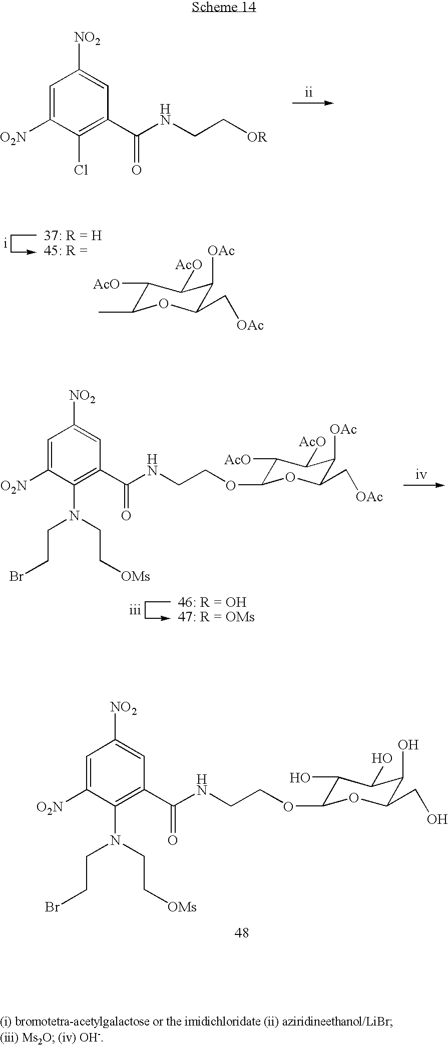

In Scheme 15, reaction of 37 with PCl₃ in pyridine gave the dimer (49) in good yield. Reaction of this with aziridineethanol, followed by mesylation, gave 51, which could be oxidised to the phosphate 52.

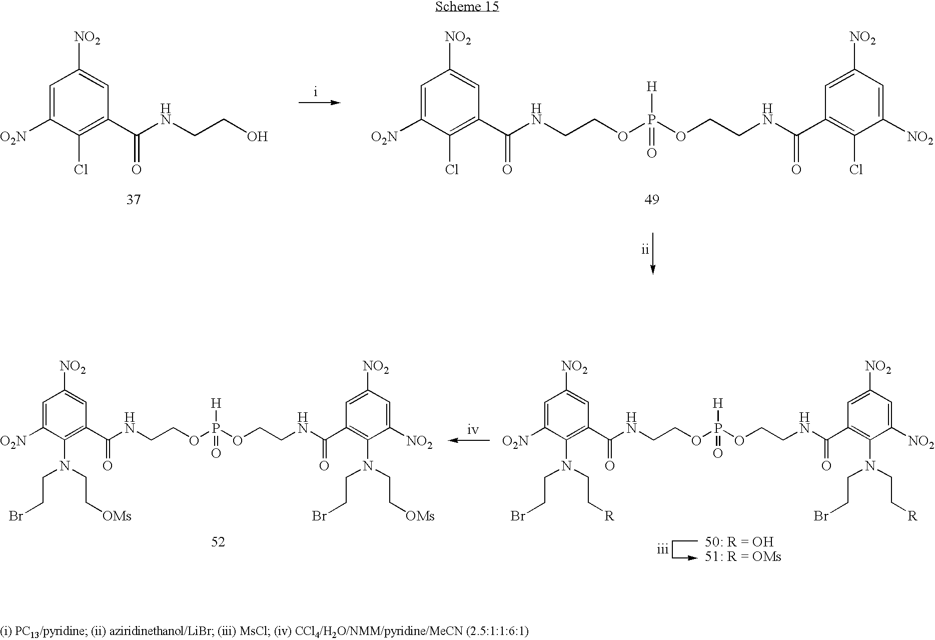

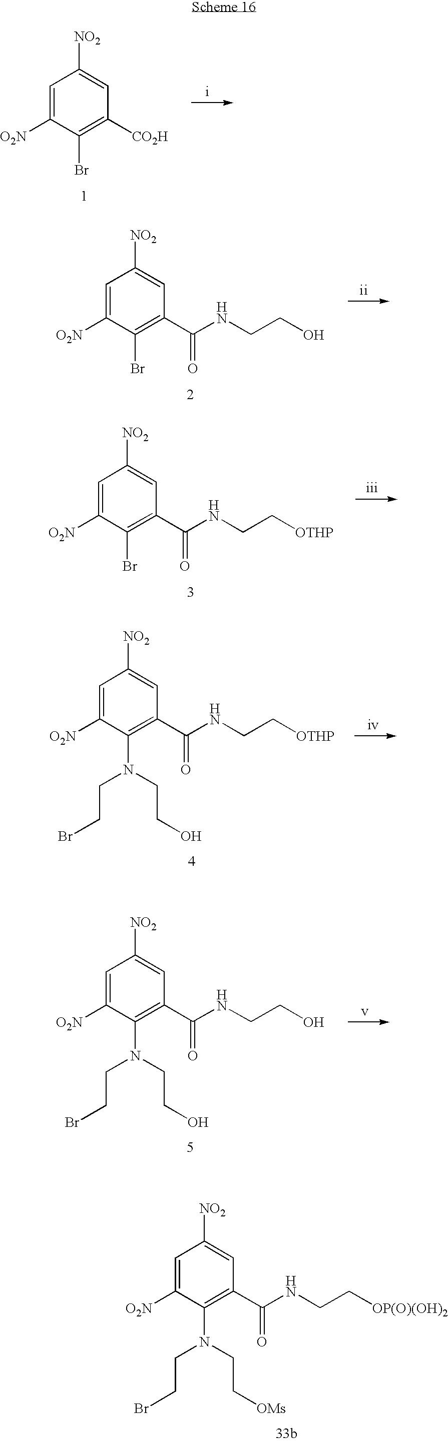

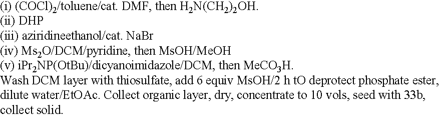

### EXAMPLES

The invention is illustrated by the following non-limiting Examples 1-21, including Examples 1-3 (carboxamides), Examples 4, 5 (alcohols), Example 6 (alkylphosphates), and Examples 7-9 (esters).

### Example 1

### Table 1, Entry 1, and Scheme 5

### Preparation of 2-[5-(aminocarbonyl)(2-chloroethyl)-2,4-dinitroanilino]ethyl methanesulfonate (10a)

A solution of 5-chloro-2,4-dinitrobenzamide [Palmer et al., J. Med. Chem., 1992, 35, 3214] (8) (2.10 g, 8.55 mmol) in dry DMF (10 mL) was treated with aziridineethanol (2.98 g, 34.2 mmol) followed by LiCl (0.36 g, 8.5 mmol) and stirred at room temperature for 6 h. The mixture was diluted with brine (40 mL) and extracted with EtOAc (2×60 mL). The combined organic fractions were washed with brine dried and concentrated under reduced pressure, chromatography on silica gel and elution with EtOAc provided a gun which was triturated with EtOAc/CH₂Cl₂ to give 5-[(2-chloroethyl)(2-hydroxyethyl)amino]-2,4-dinitrobenzamide (9a) (1.22 g, 43%) as a yellow solid: mp (EtOAc/petroleum ether) 131-134° C.; ¹H NMR [(CD₃)₂SO] δ 8.48 (s. 1H), 8.10 (s, 1H), 7.75 (s, 1H), 7.35 (s, 1H), 4.76 (vbr s, 1H), 3.83 (t, J=5.9 Hz, 2H), 3.75 (t, J=5.9 Hz, 2H), 3.56 (t, J=5.4 Hz, 2H), 3.36 (t, J=5.5 Hz, 2H); ¹³C NMR δ 166.3, 147.3, 137.4, 136.9, 135.-0, 124.3, 119.8, 58.1, 54.1, 52.5, 41.3; Anal. Calcd. for C₁₁H₁₃ClN₄O₆: C, 39.7; H, 3.9; N, 16.8. Found: C, 39.8; H, 3.9; N, 16.6%.

A stirred solution of 9a (400 mg, 1.20 mmol) in pyridine (1.5 mL) was cooled at 0° C. and then treated with MsCl (1.13 μL, 1.44 mmol) for 1.5 h. The mixture was diluted with water and extracted with EtOAc (2×50 mL). The combined organic fractions were washed with water (3 times) then dried and concentrated under reduced pressure. Chromatography on silica gel and elution with EtOAc/petroleum ether (9:1) gave 10a (364 mg, 74%) as a yellow solid, identical (¹H NMR, ¹³CNMR, HPLC) with the compound prepared previously by an alternate route [Palmer et al. J. Med. Chem., 1992, 35, 3214].

### Example 2

### Table 1, Entry 1, and Scheme 5

### Preparation of 2-[5-(aminocarbonyl)(2-bromoethyl)-2,4-dinitroanilino]ethyl methanesulfonate (10b)

A slurry of 9a (110 mg, 0.33 mmol) in dry 3-methyl-2-butanone (15 mL) and LiBr (2.0 g) was heated under reflux for 5 hrs. The reaction mixture was cooled, then water (50 mL) was added, and the mixture was extracted with EtOAc (3×50 mL). The combined organic layer was dried and concentrated under pressure, then passed through a short column of silica gel, eluting with heptane/EtOAc (1:1), to give 5-[(2-bromoethyl)(2-hydroxyethyl)amino]-2,4-dinitrobenzamide (9b) (107 mg, 86%) as a yellow solid: mp (EtOAc/petroleum ether) 144-147° C.; ¹H NMR [(CD₃)₂SO] δ 8.48 (s. 1H), 8.10 (s, 1H), 7.76 (s, 1H), 7.33 (s, 1H), 4.76 (vbr s, 1H), 3.80 (t, J=5.9 Hz, 2H), 3.69 (t, J=5.9 Hz, 2H), 3.56 (t, J=5.4 Hz, 2H), 3.36 (t, J=5.5 Hz, 2H); ¹³C NMR δ 166.2, 147.1, 137.8, 137.4, 135.1, 124.3, 119.7, 58.1, 54.0, 52.5, 29.9; Anal. Calcd. for C₁₁H₁₃BrN₄O₆: C, 35.0; H, 3.5. Found: C, 36.3; H, 3.6%.

A stirred solution of 9b (13 mg, 0.35 mmol) in THF (2.0 mL) was cooled at 0° C. and then treated with MsCl (1.13 μL, 1.44 mmol) for 1.5 h. The mixture was diluted with water and extracted with EtOAc (2×50 mL). The combined organic fractions were washed with water (3 times) then dried and concentrated under reduced pressure. Chromatography on silica gel and elution with EtOAc/petroleum ether (9:1) gave 10b (15 mg, 96%) as a yellow solid, identical (¹H NMR, ¹³CNMR, HPLC) with compound prepared previously by an alternate route [Palmer et al. J. Med. Chem., 1992, 35, 3214].

### Example 3

### Table 1, Entry 1, and Scheme 5

### Preparation of 2-[5-(aminocarbonyl)(2-iodoethyl)-2,4-dinitroanilino]ethyl methanesulfonate (10c)

A slurry of 9a (115 mg, 0.35 mmol) in dry 3-methyl-2-butanone (15 mL) and NaI (2.0 g) was heated under reflux for 5 hrs. The reaction mixture was cooled, then water (50 mL) was added, and the mixture was extracted with EtOAc (3×50 mL). The combined organic layer was dried and concentrated under pressure, then passed through a short column of silica gel, eluting with heptane/EtOAc (1:1), to give 5-[(2-iodoethyl)(2-hydroxyethyl)amino]-2,4-dinitrobenzamide (9c) (100 mg, 68%) as a yellow solid: mp (EtOAc/petroleum ether) 144-147° C.; ¹H NMR [(CD₃)₂SO] δ 8.47 (s. 1H), 8.10 (s, 1H), 7.76 (s, 1H), 7.30 (s, 1H), 4.76 (t, J=5.3 Hz, 1H), 3.74 (t, J=7.1 Hz, 2H), 3.54 (m, 2H), 3.38 (m, 4H); ¹³C NMR δ 166.2, 146.7, 137.4, 136.8, 135.0, 124.2, 119.6, 58.1, 53.9, 53.4, 2.5; Anal. Calcd. for C₁₁H₁₃IN₄O₆: C, 31.1; H, 3.1; N, 13.2. Found: C, 31.4; H, 3.6; N, 12.9%.

A stirred solution of 9c (11 mg, 0.26 mmol) in THF (2.0 mL) was cooled at 0° C. and then treated with MsCl (1.13 μL, 1.44 mmol) for 1.5 h. The mixture was diluted with water and extracted with EtOAc (2×50 mL). The combined organic fractions were washed with water (3×) then dried and concentrated under reduced pressure. Chromatography on silica gel and elution with EtOAc/petroleum ether (9:1) gave 10c (15 mg, 97%) as a yellow solid, identical (¹H NMR, ¹³CNMR, HPLC) with compound prepared previously by an alternate route [Palmer et al. J. Med. Chem., 1992, 35, 3214].

### Example 4

### Table 1, Entry 2, and Scheme 6

### Preparation 2-[2-(aminocarbonyl)(2-chloroethyl)-4,6-dinitroanilino]ethyl methanesulfonate (13a)

A solution of 2-chloro-3,5-dinitrobenzamide (11) (250 mg, 1.15 mmol) in THF (20 mL) was cooled to below 5° C., and aziridineethanol (240 mg, 2.76 mmol) was added over 10 min to the stirred mixture, which was then kept at 20° C. overnight. Water (50 mL) was added, followed by EtOAc (50 mL). The mixture was separated and aqueous phase was extracted with EtOAc (2×60 mL). The combined organic fractions were washed with brine, dried and concentrated under reduced pressure, chromatography on silica gel and elution with EtOAc/petroleum ether (1:1), to give 2-[(2-chloroethyl)(2-hydroxyethyl)amino]-3,5-dinitrobenzamide (12a) (350 mg, 92%) as a yellow solid: mp (EtOAc/petroleum ether) 96-100° C.; ¹H NMR [(CD₃)₂SO] δ 8.69 (d, J=2.8 Hz, 1H), 8.43 (s, 1H, CONH), 8.35 (d, J=2.8 Hz, 1H), 8.10 (s, 1H), 5.15 (t, J=5.6 Hz, 1H), 3.77 (m, 2H), 3.54 (m, 4H), 3.14 (m, 2H); ¹³C NMR δ 167.6, 146.7, 143.8, 139.7, 134.1, 128.2, 123.1, 57.8, 54.3, 53.6, 41.4; Anal. Calcd. for C₁₁H₁₃ClN₄O₆: C, 39.7; H, 3.9; N, 16.8. Found: C, 38.4; H, 4.4; N, 15.7%.

A solution of 12a (16 mg, 0.05 mmol) in dry THF (10 mL) was cooled in an ice-bath at 0° C. and then treated with Et₃N (0.2 mL), followed by MsCl (0.1 mL) dropwise. After stirring for 10 min. at 0° C., satd. NaHCO₃ (20 mL) was added, and after a further 30 min the aqueous phase was extracted with CH₂Cl₂ (2×50 mL). The combined organic phases were dried, concentrated under reduced pressure and filtered through a short column, eluting with EtOAc/petroleum ether (1:1), to give 13a (20 mg, 100%) as a yellow solid: mp (EtOAc/petroleum ether) 155-157° C.; ¹H NMR [(CD₃)₂SO] δ 8.74 (d, J=2.7 Hz, 1H), 8.34 (d, J=2.7 Hz, 1H), 8.19 (s, 1H), 7.99 (s, 1H), 4.29 (m, 2H), 3.73 (m, 2H), 3.48 (m, 4H), 3.15 (s, 3H); ¹³C NMR δ 167.11, 145.98, 146.34, 140.84, 136.05, 127.26, 122.22, 67.49, 54.35, 51.34, 41.36, 36.46. Anal. calcd for C₁₂H₁₅ClN₄O₈S: C, 35.1; H, 3.7; N, 13.7; Cl, 8.5. Found: C, 35.7; H, 3.9; N, 13.6; Cl, 8.7%.

### Example 5

### Table 1, Entry 2, and Scheme 6

### Preparation of 2-[2-(aminocarbonyl)(2-bromoethyl)-4,6-dinitroanilino]ethyl methanesulfonate (13b)

A slurry of 2-chloro-3,5-dinitrobenzamide (11) (237 mg, 1.1 mmol) in dry 3-methyl-2-butanone (20 mL) was cooled to below 5° C., and LiBr (1.5 g) was added, keeping the temperature below 15° C. Aziridineethanol (240 mg, 2.76 mmol) was added to the stirred mixture, which was then kept at 20° C. overnight. Water (50 mL) was added, followed by EtOAc (100 mL). The organic layer was washed with water, 10% aqueous NaBr, and then passed through a short column of silica gel, eluting with heptane/EtOAc (1:1), to give 2-[(2-bromoethyl)(2-hydroxyethyl)amino]-3,5-dinitrobenzamide (12b) (360 mg, 87%) as a yellow solid: mp (EtOAc/petroleum ether) 138-140° C.; ¹H NMR [(CD₃)₂SO] δ 8.69 (d, J=2.8 Hz, 1H), 8.43 (s, 1H), 8.35 (d, J=2.8 Hz, 1H), 8.10 (s, 1H), 5.15 (t, J=5.6 Hz, 1H), 3.61 (m, 4H), 3.53 (m, 2H), 3.14 (m, 2H); ¹³C NMR δ 167.6, 146.4, 143.8, 139.8, 134.2, 128.2, 123.0, 57.8, 54.1, 53.6, 29.8; Anal. Calcd for C₁₁H₁₃BrN₄O₆: C, 35.0; H, 3.5; N, 14.9. Found: C, 35.0; H, 3.5; N, 14.6%.

A solution of 12b (360 mg, 0.96 mmol) in dry CH₂Cl₂ (50 mL) was cooled in an ice-bath at 0° C. and then treated with Et₃N (1.0 mL), followed by MsCl (0.5 mL) dropwise. After stirring for 10 min. at 0° C., satd. NaHCO₃ (50 mL) was added, and after a further 30 min the aqueous phase was extracted with CH₂Cl₂ (2×50 mL). The combined organic phases were dried, concentrated under reduced pressure, filtered through a short column, eluted with EtOAc/petroleum ether (1:1), concentration to give 2-[2-(aminocarbonyl)(2-bromoethyl)-4,6-dinitroanilino]ethyl methanesulfonate (13b) (369 mg, 85%) as a yellow solid: mp (EtOAc/petroleum ether) 153-154° C.; ¹H NMR [(CD₃)₂SO] δ 8.74 (d, J=2.8 Hz, 1H), 8.33 (d, J=2.8 Hz, 1H), 8.19 (s, 1H), 7.99 (s, 1H), 4.29 (m, 2H), 3.60 (m, 2H), 3.49 (m, 4H), 3.14 (s, 3H); ¹³C NMR δ 167.11, 145.75, 146.37, 140.92, 136.12, 127.24, 122.20, 67.53, 54.41, 51.16, 36.46, 29.73. Anal. Calcd. for C₁₂H₁₅BrN₄O₈S: C, 31.7; H, 3.3; N, 12.3; Br, 17.6. Found: C, 32.0; H, 3.4; N, 12.2; Br, 17.7%.

### Example 6

### Table 1, Entry 2, and Scheme 6

### Preparation of 2-[2-(aminocarbonyl)(2-iodoethyl)-4,6-dinitroanilino]ethyl methanesulfonate (13c)

A slurry of 2-chloro-3,5-dinitrobenzamide (11) (221 mg, 1.0 mmol) in dry 3-methyl-2-butanone (20 mL) was cooled to below 5° C., and NaI (2.2 g) was added, keeping the temperature below 15° C. Aziridineethanol (240 mg, 2.76 mmol) was added to the stirred mixture, which was kept at 20° C. overnight. Water (50 mL) was added, followed by EtOAc (100 mL). The organic layer was washed with water, and then passed through a short column of silica gel, eluting with heptane/EtOAc (1:1), to give 2-[(2-hydroxyethyl)(2-iodoethyl)amino]-3,5-dinitrobenzamide (12c) (297 mg, 70%) as a yellow solid: mp (EtOAc/petroleum ether) 152-155° C.; ¹H NMR [(CD₃)₂SO] δ 8.69 (d, J=2.8 Hz, 1H), 8.41 (s, 1H), 8.34 (d, J=2.8 Hz, 1H), 8.10 (s, 1H), 5.13 (t, J=5.6 Hz, 1H), 3.53 (m, 4H), 3.35 (m, 2H), 3.13 (m, 2H); ¹³C NMR δ 167.5, 146.1, 143.8, 139.8, 134.2; 128.2, 123.0, 57.8, 54.8, 53.7, 2.07. Anal. Calcd. for C₁₁H₁₃IN₄O₆: C, 31.2; H, 3.1; N, 13.2. Found: C, 31.4; H, 3.0; N, 12.9%.

A solution of 12c (150 mg, 0.35 mmol) in dry CH₂Cl₂ (100 mL) was cooled in an ice-bath at 0° C. and then treated with Et₃N (1.0 mL), followed by adding MsCl (0.5 mL) dropwise. After stirring for 10 min. at 0° C., satd. NaHCO₃ (50 mL) was added, and after a further 30 min the aqueous phase was extracted with CH₂Cl₂ (2×50 mL). The combined organic phases were dried, concentrated under reduced pressure and filtered through a short column, eluting with EtOAc/petroleum ether (1:1), to give 2-[2-(aminocarbonyl)(2-iodoethyl)-4,6-dinitroanilino]ethyl methanesulfonate (13c) (186 mg, 64%) as a yellow solid: mp (EtOAc/petroleum ether) 144-147° C.; ¹H NMR [(CD₃)₂SO] δ 8.73 (d, J=2.8 Hz, 1H), 8.33 (d, J=2.8 Hz, 1H), 8.16 (s, 1H), 7.97 (s, 1H), 4.28 (m, 2H), 3.49 (m, 4H), 3.32 (m, 2H), 3.14 (s, 3H); ¹³C NMR δ 167.1, 145.4, 145.3, 140.9, 136.1, 127.2, 122.1, 67.5, 55.6, 50.7, 36.5, 2.6. Anal. Calcd. for C₁₂H₁₅IN₄O₈S: C, 28.7; H, 3.0; N, 11.2. Found: C, 29.3; H, 3.4; N, 10.7%.

### Example 7

### Table 1, Entry 3, and Scheme 6

### Preparation of 2-[2-(aminocarbonyl)(2-bromoethyl)-4,6-dinitroanilino]ethyl ethylenesulfonate (14)

A solution of 2-[(2-bromoethyl)(2-hydroxyethyl)amino]-3,5-dinitrobenzamide (12b) (754 mg, 2.0 mmol) in dry THF (100 mL) was cooled in an ice-bath at 0° C. and then treated with Et₃N (0.7 mL), followed by vinylsulfonyl chloride (316 mg, 2.5 mmol) dropwise. After stirring for 30 min. at 0° C., ice-water was added, and after a further 30 min the aqueous phase was extracted with CH₂Cl₂ (2×50 mL). The combined organic phases were dried, concentrated under reduced pressure and filtered through a short column, eluting with EtOAc/petroleum ether (2:1), to give 2-[2-(aminocarbonyl)(2-bromoethyl)-4,6-dinitroanilino]ethyl ethylenesulfonate (14) (864 mg, 93%) as a yellow solid: mp (EtOAc/petroleum ether) 117-119° C.; ¹H NMR [(CD₃)₂SO] δ 8.73 (d, J=2.8 Hz, 1H), 8.33 (d, J=2.8 Hz, 1H), 8.18 (s, 1H), 7.96 (s, 1H), 6.89 (q, J=10.1 Hz, 1H), 6.31 (d, J=9.6 Hz, 1H), 6.28 (d, J=2.8 Hz, 2H), 4.20 (t, J=5.4 Hz, 2H), 3.55 (m, 2H), 3.49 (m, 4H), 167.1, 145.7, 145.6, 141.1, 136.3, 132.1, 131.5, 127.2, 122.1, 68.2, 54.6, 512, 29.7. Anal. Calcd. for C₁₃H₁₅BrN₄O₈S: C, 33.4; H, 3.2; N, 12.0. Found: C, 33.7; H, 3.3; N, 11.9%.

### Example 8

### Table 1, Entry 4 and Scheme 7

### Preparation of 2-[(2-chloroethyl)-2,4-dinitro-6-({[2-(tetrahydro-2H-pyran-2-yloxy)ethyl]amino}carbonyl)anilino]ethyl methanesulfonate (17a)

A solution of 2-chloro-3,5-dinitro-N-[2-(tetrahydro-2H-pyran-2-yloxy)ethyl]benzamide (15) [Denny et al., PCT Int. Appl. WO 04033415] (398 mg, 1.07 mmol) in dry 3-methyl-2-butanone (20 mL) was cooled to below 5° C., and aziridineethanol (240 mg, 2.76 mmol) was then added. The reaction was kept at 20° C. overnight, then water (100 mL) was added slowly, and the mixture was extracted with EtOAc (3×50 mL). The combined organic layers were washed with water, dried and concentrated under pressure, and then passed through a short column of silica gel, eluting with heptane/EtOAc (1:1), to give 2-[(2-chloroethyl)(2-hydroxyethyl)amino]-3,5-dinitro-N-[2-(tetrahydro-2H-pyran-2-yloxy)ethyl]benzamide (16a) (485 mg, 97%) as a yellow solid: mp 106-108° C.; ¹H NMR [(CD₃)₂SO] δ 9.07 (t, J=5.4 Hz, 1H), 8.70 (d, J=2.8 Hz, 1H), 8.33 (d, J=2.8 Hz, 1H), 5.15 (t, J=5.6 Hz, 1H), 4.62 (t, J=3.7 Hz, 1H), 3.78 (m, 4H), 3.58-3.43 (m, 8H), 3.14 (m, 2H), 1.80-1.40 (m, 6H); ¹³C NMR δ 165.7, 146.8, 143.7, 139.7, 133.9, 128.4, 123.1, 98.0, 64.8, 61.5, 57.8, 54.3, 53.3, 41.4, 39.5, 30.1, 24.9, 19.1; Anal. Calcd. for C₁₈H₂₅ClN₄O₈: C, 46.9; H, 5.5; N, 12.2. Found: C, 47.1; H, 5.4; N, 12.2%.

Similarly, a suspension of 2-[(2-chloroethyl)amino]ethanol hydrochloride (2.0 g, 8.4 mmol) in 1,4-dioxane (150 mL) was treated with Et₃N (1.8 g), cooled with ice bath and then 15 (2.0 g, 5.4 mmol) was added. The reaction was kept at 20° C. overnight, then water (100 mL) was added and the mixture was extracted with EtOAc (3×150 mL). The combined organic phases were washed with pre-cooled dilute HCl, water, aqueous NaHCO₃, and brine, dried and concentrated under pressure, and then passed through a short column of silica gel, eluting with heptane/EtOAc (1:1), to give 16a (4.1 g, 37%).

A stirred solution of 16a (210 mg, 0.46 mmol) in dry CH₂Cl₂ (10 mL) was cooled in an ice-bath at 0° C. and then treated with Et₃N (0.4 mL), followed by MsCl (0.2 mL) dropwise. After stirring for 10 min. at 0° C., satd. NaHCO₃ (10 mL) was added, and after a further 30 min the aqueous phase was extracted with CH₂Cl₂ (2×20 mL). The combined organic phases were dried, concentrated under reduced pressure and filtered through a short column, eluting with EtOAc/petroleum ether (1:1), to give 2-[(2-chloroethyl)-2,4-dinitro-6-({[2-(tetrahydro-2H-pyran-2-yloxy)ethyl]amino}carbonyl)anilino]ethyl methanesulfonate (17a) (240 mg, 98%) as a yellow foam; ¹H NMR (CDCl₃) δ 8.62 (d, J=2.8 Hz, 1H), 8.55 (d, J=2.8 Hz, 1H), 7.31 (t, J=5.4 Hz, 1H), 4.55 (m, 1H), 4.37 (m, 2H), 3.90 (m, 2H), 3.70 (m, 7H), 3.53 (m, 3H), 3.01 (s, 3H), 1.86-1.45 (m, 6H); ¹³C NMR δ 165.1, 146.0, 145.6, 142.1, 136.4, 128.7, 122.8, 100.4, 67.2, 66.2, 64.1, 55.2, 52.3, 41.7, 40.5, 37.5, 30.9, 25.2, 20.5; HRMS (FAB) calcd for C₁₉H₂₈³⁵ClN₄O₁₀S [MH]⁺ m/z 539.1215; found 539.1206.

### Further Processing of 17a to Give 2-[(2-chloroethyl)-2,4-dinitro-6-[[[2-(phosphonooxy)ethyl]amino]-carbonyl]anilino]ethyl methanesulfonate (33a)

A solution of 17a (246 mg, 0.46 mmol) in MeOH (10 mL) was treated with methanesulfonic acid (1 drop), and the solution was stirred at 20° C. for 2 h. Most of solvent was removed under reduced pressure, and the reaction mixture was then partitioned between EtOAc and water (40 mL, 1:1). The aqueous phase was extracted with EtOAc (2×20 mL), and the combined organic phases were washed with water, dried over Na₂SO₄, and concentrated under reduced pressure. The residue was purified by chromatography on silica gel, eluting with EtOAc/petroleum ether (from 1:1 to 1:0) to give 2-((2-chloroethyl)-2-{[(2-hydroxyethyl)amino]carbonyl}-4,6-dinitroanilino)ethyl methanesulfonate (31a) (198 mg, 95) as a yellow oil: ¹H NMR [(CD₃)₂SO] δ 8.77 (m, 1H, CONH), 8.74 (d, J=2.7 Hz, 1H, H-4), 8.36 (d, J=2.7 Hz, 1H, H-6), 4.28 (m, 2H, —CH₂O-Ms), 3.58 (m, 4H), 3.44 (m, 4H), 3.14 (s, 3H, —OSO₂CH₃); ¹³C NMR δ 165.3, 145.8, 145.2, 140.9, 135.1, 127.5, 122.2, 67.5, 59.2, 54.2, 51.0, 42.1, 36.4, 29.7; HRMS (FAB) calcd for C₁₄H₂₀³⁵ClN₄O₉S [MH]⁺ m/z 455.0640; found 455.0638.

A solution of 31a (2.50 g, 5.5 mmol) and di-tert-butyl diethylphosphoramidite (93%, 1.80 mL, 7.15 mmol) in dry DMF (30 mL) under N₂ was treated with 1H-tetrazole (3 wt. % in CH₃CH, 33 mL, 11.0 mmol) and stirred at 20° C. for 30 min. The reaction mixture was then cooled to −50° C. and a solution of 3-chloroperoxybenzoic acid (55%, 2.68 g, 8.0 mmol) in CH₂Cl₂ was rapidly added. The reaction mixture was warmed to room temperature and diluted with EtOAc (500 mL). The solution was washed with 5% aqueous Na₂S₂O₅ (2×50 mL), 10% aqueous NaHCO₃ (2×50 mL), water (2×50 mL), dried, concentrated under reduced pressure below 30° C. and the residue was purified by chromatography on silica gel, eluting with EtOAc/petroleum ether (from 1:1 to 1:0) to give 2-[(2-chloroethyl)-2-(6-tert-butoxy-8,8-dimethyl-6-oxido-5,7-dioxa-2-aza-6-phosphanon-1-anoyl)-4,6-dinitroanilino]ethyl methanesulfonate (32a) (80%) as a yellow foam; ¹H NMR [(CD₃)₂SO] δ 8.94 (t, J=5.6 Hz, 1H), 8.75 (d, J=2.8 Hz, 1H), 8.34 (d, J=2.8 Hz, 1H), 4.28 (t, J=5.4 Hz, 2H), 4.02 (q, J=6.2 Hz, 2H), 3.74-3.43 (m, 8H), 3.13 (s, 3H), 1.43 (s, 18H). ¹³C NMR δ 265.6, 146.2, 145.3, 140.8, 135.6, 127.5, 122.4, 81.7, 67.5, 64.2, 54.3, 51.3, 41.4, 36.5, 29.5; HRMS (FAB) calcd for C₂₂H₃₇³⁵ClN₄O₁₂SP [MH]⁺ m/z 647.1555; found 647.1555

A solution of 32a (1.70 g, 2.6 mmol) and TFA (25 mL) in dry CH₂Cl₂ (25 mL) was stirred at 20° C. for 1 h, then concentrated under reduced pressure. Residual TFA was removed azeotropically with CH₂Cl₂ (2×) and the resulting residue was dissolved in EtOAc. Addition of excess CH₂Cl₂ gave 2-[(2-chloroethyl)-2,4-dinitro-6-[[[2-(phosphonooxy)ethyl]amino]-carbonyl]anilino]ethyl methanesulfonate (33a) (68%) as a yellow solid: mp (EtOAc/CH₂Cl₂) 132-134° C.; ¹H NMR [(CD₃)₂SO] δ 8.92 (t, J=5.6 Hz, 1H), 8.74 (d, J=2.8 Hz, 1H), 8.37 (d, J=2.8 Hz, 1H), 4.29 (t, J=5.4 Hz, 2H), 3.98 (q, J=6.0 Hz, 2H), 3.58-3.40 (after D₂O exchange, m, 8H), 3.13 (s, 2H). ¹³C NMR δ 165.5, 146.1, 145.3, 140.8, 135.7, 127.6, 122.3, 67.5, 63.3, 63.2, 54.3, 51.3, 41.3, 36.5. Anal. Calcd. for C₁₄H₂₀ClN₄O₁₂PS: C, 31.4; H, 3.8; N, 10.5. Found: C, 31.7; H, 3.9; N, 10.5%.

### Example 9

### Table 1, Entry 4 and Scheme 7

### Preparation of 2-[(2-bromoethyl)-2,4-dinitro-6-({[2-(tetrahydro-2H-pyran-2-yloxy)ethyl]amino}carbonyl)anilino]ethyl methanesulfonate (17b)

A slurry of 2-chloro-3,5-dinitro-N-[2-(tetrahydro-2H-pyran-2-yloxy)ethyl]benzamide (15) (10.0 g, 24 mmol) and LiBr (40 g, 19 equiv) in dry 3-methyl-2-butanone (100 mL) was cooled to below 5° C. Aziridineethanol (5.0 g, 58 mmol) was added over 10 min to the stirred mixture, which was kept below 20° C. overnight. Water (200 mL) was added, and the reaction mixture was extracted with EtOAc (3×50 mL). The combined organic layers were washed with water, 10% aqueous NaBr, dried and concentrated under pressure, and then passed through a short column of silica gel, eluting with heptane/EtOAc (1:1), to give 2-[(2-bromoethyl)(2-hydroxyethyl)amino]-3,5-dinitro-N-[2-(tetrahydro-2H-pyran-2-yloxy)ethyl]benzamide (16b) (10.86 g, 90%) as a yellow solid: mp 121-123° C.; ¹H NMR [(CD₃)₂SO] δ 9.06 (t, J=5.4 Hz, 1H), 8.70 (d, J=2.8 Hz, 1H), 8.33 (d, J=2.8 Hz, 1H), 5.15 (t, J=5.5 Hz, 1H), 4.62 (t, J=3.5 Hz, 1H), 3.78 (m, 2H), 3.63-3.43 (m, 10H), 3.15 (m, 2H), 1.80-1.40 (m, 6H); ¹³C NMR δ 165.7, 146.6, 143.8, 139.8, 134.0, 128.4, 123.0, 98.0, 64.8, 61.5, 57.9, 54.1, 53.4, 39.5, 30.1, 29.8, 24.9, 19.1; Anal. Calcd. for C₁₈H₂₅BrN₄O₈: C, 42.8; H, 5.0; N, 11.1. Found: C, 42.8; H, 5.0; N, 11.0%.

A stirred solution of alcohol 16b (98% pure) (51.5 g, 0.10 mol) in dry CH₂Cl₂ (300 mL) containing dry pyridine (21.4 mL, 0.26 mol) was cooled to 0° C. and treated over a 5 min period with a solution of methanesulfonic anhydride (97%, 23.8 g, 0.13 mol) in CH₂Cl₂ (80 mL). The mixture was stirred at 20° C. for 1.5 h, then 10% aqueous KHCO₃ (200 mL) was added and the mixture was stirred vigorously for 30 min, then concentrated under reduced pressure to remove all of the CH₂Cl₂. The oil that precipitated was separated from the remaining liquid, rinsed with water, and dissolved in EtOAc (300 mL). The EtOAc solution was washed with water (2×), dried, and filtered through a short column of silica gel. The eluate was concentrated to 100 mL and shaken with excess hexane (250 mL). The precipitated oil was separated from the mother liquor and dried, to give mesylate 17b as yellow foam (56.4 g, 95%) (98.3% pure); ¹H NMR (CDCl₃) δ 8.62 (d, J=2.8 Hz, 1H), 8.53 (d, J=2.8 Hz, 1H), 7.29 (br, 1H), 4.55 (m, 1H), 4.37 (m, 2H), 3.90 (m, 2H), 3.78 (m, 1H), 3.73-3.49 (m, 9H), 3.01 (s, 3H), 1.90-1.48 (m, 6H); ¹³C NMR δ 165.0, 145.9, 145.6, 142.1, 136.4, 128.5, 122.8, 100.5, 67.1, 66.3, 64.2, 55.4, 52.1, 40.5, 37.5, 31.0, 29.0, 25.2, 20.6; HRMS (FAB) calcd for C₁₉H₂₈⁷⁹BrN₄O₁₀S [MH]⁺ m/z 583.0710; found 583.0712.

### Further Processing of 17b to Give 2-[(2-bromoethyl)-dinitro-6-[[[2-(phosphonooxy)ethyl]amino]-carbonyl]anilino]ethyl methanesulfonate (33b)

A solution of 17b (54.8 g, 94 mmol) in MeOH (1500 mL) was treated with methanesulfonic acid (1.5 ml), and the solution was stirred at 20° C. for 2 h. Most of the solvent was removed under reduced pressure, and the reaction mixture was then partitioned between EtOAc and water (1000 mL, 1:1). The aqueous phase was extracted with EtOAc (2×300 mL), and the combined organic phases were washed with water, dried over Na₂SO₄, and concentrated under reduced pressure. The residue was purified by chromatography on silica gel, eluting with EtOAc/petroleum ether (from 1:1 to 1:0), to give 2-((2-bromoethyl)-2-{[(2-hydroxyethyl)amino]carbonyl}-4,6-dinitroanilino)ethyl methanesulfonate (31b) as a yellow oil (43.8 g, 93%); ¹H NMR [(CD₃)₂SO] δ 8.77 (m, 1H, CONH), 8.74 (d, J=2.7 Hz, 1H, H-4), 8.36 (d, J=2.7 Hz, 1H, H-6), 4.28 (m, 2H, CH₂OMs), 3.58 (m, 4H), 3.44 (m, 4H), 3.14 (s, 3H, OSO₂CH₃); ¹³C NMR δ 165.3, 145.8, 145.3, 140.9, 136.2, 127.5, 122.1, 67.5, 59.2, 54.3, 51.0, 42.1, 36.5, 29.7; HRMS (FAB) required for C₁₄H₂₀⁷⁹BrN₄O₉S [MH⁺] m/z 499.01344; Found 499.01324.

A solution of 1H-tetrazole (162 mL, 55 mmol, 3 wt % in CH₃CN) was concentrated under reduced pressure below 30° C. to a moist solid (CAUTION; POTENTIALLY EXPLOSIVE) and a solution of 31b (17.15 g, 34.3 mmol, 95% pure) in dry N,N-dimethylformamide (60 mL) was then added. The mixture was stirred at 20° C. (cooling) under N₂ and treated slowly over a 25 min period with di-tert-butyl diisopropylphosphoramidite (14.8 mL, 44.6 mmol). After stirring at 20° C. for a further 2.0 h, the reaction mixture was cooled and treated slowly over a 10 min period at 0° C. with a solution of 70% aqueous H₂O₂ (8.5 mL) in THF (10 mL). The mixture was allowed to warm to 10° C. for 45 min, then poured into cold 2% aqueous Na₂S₂O₅ (2 L) and refrigerated. The precipitated oil was separated from the mother liquors and dissolved in EtOAc (2000 ml). The solution was washed with water, dried, concentrated (below 30° C.) and the residue was purified by chromatography on silica gel, eluting with EtOAc/petroleum ether (9:1). The product-containing fractions were concentrated to a small volume and diluted with excess hexane to precipitate an oil. This was dried under high vacuum to provide 2-[(2-bromoethyl)-2-(6-tert-butoxy-8,8-dimethyl-6-oxido-5,7-dioxa-2-aza-6-phosphanon-1-anoyl)-4,6-dinitroanilino]ethyl methanesulfonate (32b) (15.64 g, 66%) as a yellow foam; ¹H NMR [(CD₃)₂SO] δ 8.94 (t, J=5.6 Hz, 1H), 8.75 (d, J=2.8 Hz, 1H), 8.34 (d, J=2.8 Hz, 1H), 4.28 (t, J=5.4 Hz, 2H), 4.02 (q, J=6.2 Hz, 2H), 3.62-3.43 (m, 8H), 3.13 (s, 3H), 1.43 (s, 18H). HRMS (FAB) calcd for C₂₂H₃₇⁷⁹BrN₄O₁₂PS [MH]⁺ m/z 693.1029; found 693.1010.

A solution of 32b (14.5 g, 21.0 mmol, 95% pure) in dry CH₂Cl₂ (40 mL) was treated with trifluoroacetic acid (60 mL) and stirred at 20° C. for 30 min. The solution was evaporated under reduced pressure (water pump) below 30° C. then under high vacuum for 45 min. This was dissolved in dry acetonitrile (25 mL). The solution was diluted with dry CH₂Cl₂ until cloudy, seeded, and then refrigerated (5° C.) for 16 h. The separated solid was collected, washed with acetonitrile/CH₂Cl₂ (2:1), CH₂Cl₂, and hexane and then dried under high vacuum to give 2-[(2-bromoethyl)-2,4-dinitro-6-[[[2-(phosphonooxy)ethyl]amino]-carbonyl]anilino]ethyl methanesulfonate (33b) (11.4 g, 91% yield, 97.4% pure) as a yellow solid: mp 135-137° C.; ¹H NMR [(CD₃)₂SO] δ 8.93 (t, J=5.6 Hz, 1H), 8.75 (d, J=2.8 Hz, 1H), 8.36 (d, J=2.8 Hz, 1H), 4.29 (t, J=5.6 Hz, 2H), 3.97 (q, J=6.3 Hz, 2H), 3.61-3.55 (m, 2H), 3.55-3.43 (m, 6H), 3.13 (s, 3H); ¹³C NMR 165.6, 146.0, 145.3, 140.9, 135.8, 127.6, 122.4, 67.6, 63.4, 63.3, 54.4, 51.2, 39.9, 36.5, 29.8. HRMS (FAB) calcd for C₁₃H₁₈⁷⁹Br₂N₄O₉P (MH⁺) m/z 562.9178, found 562.9171; calcd for C₁₃H₁₈⁷⁹Br^{8l}BrN₄O₉P (MH⁺) m/z 564.9158, found 564.9152; calcd for C₁₃H₁₈⁸¹Br₂N₄O₉P. (MH⁺) m/z 566.9137, found 566.9121; Anal. Calcd. for C₁₄H₂₀BrN₄O₁₂PS: C, 29.0; H, 3.5; N, 9.7. Found: C, 29.1; H, 3.3; N, 9.6%.

### Example 10

### Table 1, Entry 4 and Scheme 7

### Preparation of 2-[(2-iodoethyl)-2,4-dinitro-6-({[2-(tetrahydro-2H-pyran-2-yloxy)ethyl]amino}carbonyl)anilino]ethyl methanesulfonate (17c)

A slurry of 15 (520 mg, 1.4 mmol) in dry 3-methyl-2-butanone (20 mL) and NaI (3.1 g) was cooled to below 5° C., and aziridineethanol (240 mg, 2.76 mmol) was added. The reaction was kept at 20° C. overnight, then water (100 mL) was added, and the mixture was extracted with EtOAc (3×50 mL). The combined organic layer was washed with water, dried and concentrated under pressure, then passed through a short column of silica gel, eluting with heptane/EtOAc (1:1), to give 2-[(2-hydroxyethyl)(2-iodoethyl)amino]-3,5-dinitro-N-[2-(tetrahydro-2H-pyran-2-yloxy)ethyl]benzamide (16c) (630 mg, 82%) as a yellow foam; ¹H NMR (CDCl₃) δ 8.63 (d, J=2.8 Hz, 1H), 8.56 (d, J=2.8 Hz, 1H), 8.24 (br, 1H), 4.54 (m, 1H), 4.48 (m, 1H), 3.93 (m, 2H), 3.84 (m, 1H), 3.70 (m, 6H), 3.54 (m, 1H), 3.30 (m, 2H), 3.18 (m, 2H), 1.90-1.40 (m, 6H); ¹³C NMR δ 165.7, 146.7, 144.4, 140.9, 134.5, 129.2, 123.5, 101.6, 67.0, 65.4, 58.0, 55.0, 53.7, 40.7, 31.3, 25.1, 21.2, 0.3; HRMS (FAB) calcd for C₁₈H₂₆IN₄O₈ [MH]⁺ m/z 553.0795; found 533.0797.

A stirred solution of 16c (177 mg, 0.32 mol) in dry CH₂Cl₂ (50 mL) was cooled in an ice-bath at 0° C. and then treated with Et₃N (0.6 mL), followed by MsCl (0.3 mL) dropwise. After stirring for 10 min. at 0° C., satd. NaHCO₃ (20 mL) was added, and after a further 30 min the aqueous phase was extracted with CH₂Cl₂ (2×50 mL). The combined organic phases were dried, concentrated under reduced pressure and filtered through a short column, eluting with EtOAc/petroleum ether (1:1), to give 17c (200 mg, 99%) as a yellow foam; ¹H NMR (CDCl₃) δ 8.62 (d, J=2.8 Hz, 1H), 8.52 (d, J=2.8 Hz, 1H), 7.21 (br, 1H), 4.55 (m, 1H), 4.35 (m, 2H), 3.90 (m, 2H), 3.80 (m, 1H), 3.70-3.5 (m, 7H), 3.30 (m, 2H), 3.00 (s, 3H), 1.90-1.50 (m, 6H); ¹³C NMR δ 165.0, 145.7, 142.0, 136.3, 128.2, 122.8, 100.6, 66.9, 66.5, 64.3, 56.7, 51.6, 40.5, 37.6, 31.0, 25.2, 20.6, 0.7; HRMS (FAB) calcd for C₁₉H₂₈IN₄O₁₀S [MH]⁺ m/z 631.0571; found 631.0575.

### Further Processing of 17c to Give 2-[(2-iodoethyl)-2,4-dinitro-6-({[2-(phosphonooxy)ethyl]amino}carbonyl)-anilino]ethyl methanesulfonate (33c)

A solution of 17c (30 mg, 0.05 mmol) in MeOH (5 mL) was treated with methanesulfonic acid (1 drop), and the solution was stirred at 20° C. for 2 h. Most of the solvent was removed under reduced pressure, and the reaction mixture was then partitioned between EtOAc and water (20 mL, 1:1). The aqueous phase was extracted with EtOAc (2×10 mL), and the combined organic phases were washed with water, dried over Na₂SO₄, and concentrated under reduced pressure. The residue was purified by chromatography on silica gel, eluting with EtOAc/petroleum ether (from 1:1 to 1:0) to give 2-((2-iodoethyl)-2-{[(2-hydroxyethyl)amino]carbonyl}-4,6-dinitroanilino)ethyl methanesulfonate (31c) (25 mg, 96%) as a yellow oil ¹H NMR [(CD₃)₂SO] δ 8.74 (d, J=2.8 Hz, 1H), 8.74 (m, 1H), 8.34 (d, J=2.8 Hz, 1H), 4.28 (m, 2H), 3.56 (m, 2H), 3.43 (m, 2H), 3.31 (m, 6H), 3.13 (s, 3H); ¹³C NMR δ 165.3, 145.5, 145.2, 140.8, 136.1, 127.4, 122.1, 67.5, 59.2, 55.4, 50.6, 42.1, 36.5, 2.6. HRMS (FAB) Calcd. For C₁₄H₂₀IN₄O₉S [MH⁺] m/z 546.9996. Found; 546.9997.

Phosphorylation of 31c (1.68 g, 3.1 mmol) as above (see Example 6) with di-tert-butyl diethylphosphoramidite (93%, 1.15 g, 4.5 mmol), followed by flash column chromatography on silica gel, eluting with EtOAc/petroleum ether (1:1), and crystallization from EtOAc/petroleum ether, gave 2-[2-(6-tert-butoxy-8,8-dimethyl-6-oxido-5,7-dioxa-2-aza-6-phosphanon-1-anoyl)(2-iodoethyl)-4,6-dinitroanilino]ethyl methanesulfonate (32c) as a yellow solid (2.23 g, 97%): mp (EtOAc/petroleum ether) 109-111° C.; ¹H NMR [(CD₃)₂SO] δ 8.98 (m, 1H), 8.76 (d, J=2.8 Hz, 1H), 8.33 (d, J=2.8 Hz, 1H), 4.27 (m, 2H), 4.00 (m, 2H), 3.53 (m, 2H), 3.46 (m, 4H), 3.14 (s, 3H), 1.43 (s, 18H). ¹³C NMR δ 165.5, 145.6, 145.2, 140.8, 135.6, 127.4, 122.4, 81.7, 67.5, 64.2, 55.4, 50.7, 39.9, 36.5, 29.3, 2.6. Anal. Calcd. for C₂₂H₃₆IN₄O₁₂PS: C, 35.8; H, 4.9; N, 7.6. Found: C, 35.9; H, 5.0; N, 8.6%.

Hydrolysis of 32c (405 mg) as above (see Example 6) with trifluoroacetic acid (6 mL) and crystallization of the product from CH₂Cl₂/petroleum ether, gave 2-[(2-iodoethyl)-2,4-dinitro-6-({[2-(phosphonooxy)ethyl]amino}carbonyl)-anilino]ethyl methanesulfonate (33c) as a yellow solid (306 mg, 89%): mp 147-150° C.; ¹H NMR [(CD₃)₂SO] δ 8.93 (m, 1H), 8.74 (d, J=2.8 Hz, 1H), 8.36 (d, J=2.8 Hz, 1H), 4.27 (m, 2H), 4.00 (m, 2H), 3.46 (m, 6H), 3.31 (m, 2H), 3.12 (s, 3H). ¹³C NMR δ 165.5, 145.6, 145.2, 140.8, 135.7, 127.6, 122.3, 67.6, 63.3, 55.5, 50.7, 39.9, 36.5, 2.7. Anal. Calcd. for C₁₄H₂₀IN₄O₉PS: C, 26.8; H, 3.2; N, 8.9. Found: C, 26.9; H, 3.2; N, 8.7%.

### Example 11

### Table 1, Entry 5, and Scheme 8

### Preparation of 2-[(2-bromoethyl)-2,4-dinitro-6-({[2-(tetrahydro-2H-pyran-2-yloxy)ethyl]amino}carbonyl)anilino]ethyl 1-butanesulfonate (18)

A stirred solution of 16b (5.0 g, 9.9 mmol) in dry CH₂Cl₂ (250 mL) was cooled in an ice-bath at 0° C. and then treated with Et₃N (5.0 mL), followed by 1-butanesulfonyl chloride (4.6 g, 3.0 mmol) dropwise. After stirring for 10 min. at 0° C., satd. NaHCO₃ (50 mL) was added, and after a further 30 min the aqueous phase was extracted with CH₂Cl₂ (2×100 mL). The combined organic phases were dried, concentrated under reduced pressure, filtered through a short column, eluted with EtOAc/petroleum ether (1:1), concentration to give 2-[(2-bromoethyl)-2,4-dinitro-6-({[2-(tetrahydro-2H-pyran-2-yloxy)ethyl]amino}carbonyl)anilino]ethyl 1-butanesulfonate (18) (6.2 g, 100%) as a yellow foam; ¹H NMR [(CD₃)₂SO] δ 8.80 (t, J=5.6 Hz, 1H), 8.75 (d, J=2.8 Hz, 1H), 8.32 (d, J=2.8 Hz, 1H), 4.60 (t, J=3.3 Hz, 1H), 4.28 (t, J=5.5 Hz, 2H), 3.79 (m, 2H), 3.59 (m, 3H), 3.48 (m, 7H), 3.28 (m, 2H), 1.75 (m, 1H), 1.62 (m, 3H), 1.48 (m, 4H), 1.35 (m, 2H), 0.86 (t, J=7.3 Hz, 3H); ¹³C NMR δ 165.3, 145.8, 145.3, 140.9, 136.0, 127.4, 122.1, 98.2, 67.1, 64.9, 61.6, 54.3, 51.2, 48.5, 30.1, 29.6, 24.8, 20.5, 19.2, 13.2; HRMS (FAB) calcd for C₂₂H₃₄⁷⁹BrN₄O₁₀S [MH]⁺ m/z 625.1179; found 625.1159.

### Further Processing of 18 to Give 2-[(2-bromoethyl)-2,4-dinitro-6-({[2-(phosphonooxy)ethyl]amino}carbonyl)anilino]ethyl 1-butanesulfonate (36)

A solution of 18 (6.2 g, 9.9 mmol) in MeOH (250 mL) was treated with methanesulfonic acid (0.5 ml), and the solution was stirred at 20° C. for 2 h. Most of the solvent was removed under reduced pressure, and the reaction mixture was then partitioned between EtOAc and water (300 mL, 1:1). The aqueous phase was extracted with EtOAc (2×100 mL), and the combined organic phase were washed with water, dried over Na₂SO₄, and concentrated under reduced pressure. The residue was purified by chromatography on silica gel, eluting with EtOAc/petroleum ether (from 1:1 to 1:0) to give 2-((2-bromoethyl)-2-{[(2-hydroxyethyl)amino]carbonyl}-4,6-dinitroanilino)ethyl 1-butanesulfonate (34) as a yellow foam (4.77 g, 89%); ¹H NMR [(CD₃)₂SO] δ 8.74 (d, J=2.8 Hz, 1H), 8.72 (t, J=5.6 Hz, 1H), 8.36 (d, J=2.8 Hz, 1H), 4.80 (br, 1H), 4.28 (t, J=5.5 Hz, 2H), 3.58 (m, 4H), 3.48 (m, 4H), 3.37 (m, 2H), 3.28 (m, 3H), 1.60 (m, 2H), 1.35 (m, 2H), 0.87 (t, J=7.3 Hz, 3H); ¹³C NMR δ 165.3, 145.8, 145.3, 140.9, 136.3, 127.4, 122.1, 67.2, 59.2, 54.3, 51.2, 48.5, 42.1, 29.6, 24.8, 20.5, 13.2; HRMS (FAB) calcd for C₁₇H₂₆⁷⁹BrN₄O₉S [MH]⁺ m/z 541.0604; found 541.0596.

A solution of alcohol 34 (4.77 g, 8.8 mmol) and di-tert-butyl diisopropylphosphoramidite (96%, 3.2 mL, 13 mmol) in dry DMF (40 mL) under N₂ was treated with 1H-tetrazole (3 wt. % in CH₃CN, 48 mL, 16 mmol) and stirred at 20° C. for 1.5 h. The reaction mixture was treated with 2.5 mL of 70% H₂O₂ for 30 min. at room temperature, then poured into cold 2% aqueous Na₂S₂O₅ (500 mL) and refrigerated. The precipitated oil was separated from the mother liquors and dissolved in EtOAc (200 ml). The solution was washed with water, dried, concentrated (below 30° C.) and the residue was purified by chromatography on silica gel, eluting with EtOAc/petroleum ether (9:1). The product-containing fractions were concentrated to a small volume and diluted with excess hexane to precipitate an oil. This was dried under high vacuum to provide 2-[(2-bromoethyl)-2-(6-tert-butoxy-8,8-dimethyl-6-oxido-5,7-dioxa-2-aza-6-phosphanon-1-anoyl)-4,6-dinitroanilino]ethyl 1-butanesulfonate (35) as a yellow foam; ¹H NMR [(CD₃)₂SO] δ 8.93 (t, J=5.6 Hz, 1H), 8.76 (d, J=2.8 Hz, 1H), 8.35 (d, J=2.8 Hz, 1H), 4.28 (t, J=5.5 Hz, 1H), 4.02 (m, 2H), 3.58 (m, 4H), 3.45 (m, 4H), 3.28 (m, 2H), 1.60 (m, 2H), 1.43 (s, 18H), 1.35 (m, 2H), 0.86 (t, J=7.4 Hz, 3H). ¹³C NMR δ 165.4, 145.9, 145.4, 140.8, 135.7, 127.4, 122.3, 81.6, 67.2, 64.1, 54.3, 51.3, 48.5, 39.6, 29.7, 29.3, 24.8, 20.5, 13.2. HRMS (FAB) calcd for C₂₉H₄₀⁷⁹BrN₄O₁₂PS [MH]⁺ m/z 733.1519; found 733.1529.

A solution of 35 (4.2 g, 5.7 mmol) in dry CH₂Cl₂ (30 mL) was treated with trifluoroacetic acid (30 mL) and stirred at 20° C. for 30 min. The solution was evaporated under reduced pressure (water pump) below 30° C., then under high vacuum for 45 min. This was dissolved in dry acetonitrile (15 mL). The solution was diluted with dry CH₂Cl₂ until cloudy, and then refrigerated (5° C.) for 16 h. The separated solid was collected, washed with acetonitrile/CH₂Cl₂ (2:1), CH₂Cl₂, and hexane and then dried under high vacuum to give 2-[(2-bromoethyl)-2,4-dinitro-6-({[2-(phosphonooxy)ethyl]amino}carbonyl)anilino]ethyl 1-butanesulfonate (36) (3.17 g, 90%) as a yellow solid: mp 123-125° C.; ¹H NMR [(CD₃)₂SO] δ 8.91 (t, J=5.6 Hz, 1H), 8.75 (d, J=2.8 Hz, 1H), 8.37 (d, J=2.8 Hz, 1H), 4.28 (t, J=5.4 Hz, 2H), 3.98 (q, J=6.3 Hz, 2H), 3.58 (m, 2H), 3.50 (m, 6H), 3.26 (m, 2H), 1.60 (m, 2H), 1.36 (m, 2H), 0.86 (t, J=7.4 Hz, 3H); ¹³C NMR 165.4, 145.8, 145.4, 140.9, 135.8, 127.5, 122.2, 67.2, 63.3, 63.2, 54.3, 51.3, 48.5, 39.8, 29.8, 24.8, 20.5, 13.2; Anal. Calcd. for C₁₇H₂₆BrN₄O₁₂PS: C, 32.9; H, 4.2; N, 9.0. Found: C, 32.9; H, 4.2; N, 9.0%.

### Example 12

### Table 1, Entry 6, and Scheme 9

### Preparation of di(tert-butyl) 2-({2-[(2-chloroethyl)(2-hydroxyethyl)amino]-3,5-dinitrobenzoyl}amino)ethyl phosphate (21)

A solution of 2-chloro-3,5-dinitro-N-(2-hydroxyethyl)benzamide 37 (2.00 g, 6.91 mmol) in DMF (8 mL) and THF (12 mL) was treated at 10° C. with 1H-tetrazole (0.77 g, 11.0 mmol) followed by di-tert-butyl diisopropylphosphoramidite (95%, 2.62 g, 9.44 mmol). After being stirred at room temperature for 2 h under N₂ the reaction mixture was treated with a solution of 70% aqueous H₂O₂ (1.8 mL) in THF (2.0 mL) at 10° C. for 30 min and then diluted with 5% aqueous Na₂S₂O₅ (200 mL). The oil that precipitated was separated from the liquid and dissolved in EtOAc and the solution was washed with water, dried and concentrated under reduced pressure below 30° C., the residue was purified by chromatography on silica gel, eluting with EtOAc/petroleum ether (4:1), followed by recrystallisation from EtOAc/diisopropyl ether to give di(tert-butyl) 2-[(2-chloro-3,5-dinitrobenzoyl)amino]ethyl phosphate (19) (1.69 g, 51%) as a white solid: mp 98° C. (dec); ¹H NMR [(CD₃)₂SO] δ 9.05-8.98 (m, 2H), 8.53 (d, J=2.6 Hz, 1H), 4.00 (q, J=6.1 Hz, 2H), 3.53 (q, 5.6 Hz, 2H), 1.43 (s, 18H). ¹³C NMR δ 163.0, 148.5, 145.8, 139.8, 128.3, 125.8, 120.7, 81.5 (d, J=7.1 Hz, 2), 61.2 (d, J=6.4 Hz), 39.5 (d, J=8.5 Hz), 29.3 (d, J=4.3 Hz, 6H). Anal. Calcd. for C₁₇H₂₃ClN₃O₉P: C, 42.4; H, 5.2; N, 8.7; P, 6.4. Found: C, 42.7; H, 5.2; N, 9.0; P, 6.4%%.

A suspension of 2-[(2-chloroethyl)amino]ethanol hydrochloride (1.0 g) Et₃N (2.0 g) in 1,4-dioxane was cooled in an ice bath, and 19 (478 mg, 0.99 mmol) was added. The mixture was stirred at room temperature overnight, then water (100 mL) was added and the mixture was extracted with EtOAc (3×100 mL). The combined organic layers were washed with water, dried and concentrated under pressure, and then passed through a short column of silica gel, eluting with heptane/EtOAc (from 1:1 to 0:1), to give di(tert-butyl) 2-({2-[(2-chloroethyl)(2-hydroxyethyl)amino]-3,5-dinitrobenzoyl}amino)ethyl phosphate (20) as a yellow foam (510 mg, 90%); ¹H NMR [(CD₃)₂SO] δ 8.94 (t, J=5.5 Hz, 1H), 8.71 (d, J=2.8 Hz, 1H), 8.34 (d, J=2.8 Hz, 1H), 5.13 (t, J=5.6 Hz, 1H), 4.02 (m, 3H), 3.77 (m, 2H), 3.55 (m, 6H), 3.14 (m, 2H), 1.42 (s, 18H). ¹³C NMR δ 266.0, 146.9, 143.7, 139.6, 133.6, 128.3, 123.2, 81.6, 64.0, 59.6, 54.3, 53.3, 41.5, 29.3. HRMS (FAB) calcd for C₂₁H₃₅³⁵ClN₄O₁₀S [MH]⁺ m/z 569.1779; Found 569.1772.

### Further Processing of 20 to Give 2-[(2-chloroethyl)-2-(6-tert-butoxy-8,8-dimethyl-6-oxido-5,7-dioxa-2-aza-6-phospha non-1-anoyl)-4,6-dinitroanilino]ethyl methanesulfonate (25)

A stirred solution of 20 (470 mg, 0.82 mmol) in dry CH₂Cl₂ (30 mL) was cooled in an ice-bath at 0° C. and then treated with Et₃N (1.0 mL), followed by MsCl (0.5 mL) dropwise. The reaction was stirred for 10 min. at 0° C., then satd. NaHCO₃ (10 mL) was added, and after a further 30 min the aqueous phase was extracted with CH₂Cl₂ (2×20 mL). The combined organic phases were dried, concentrated under reduced pressure, filtered through a short column, eluted with EtOAc/petroleum ether (from 1:1 to 1:0), to give 2-[(2-chloroethyl)-2-(6-tert-butoxy-8,8-dimethyl-6-oxido-5,7-dioxa-2-aza-6-phosphanon-1-anoyl)-4,6-dinitroanilino]ethyl methanesulfonate (21) (400 mg, 75%) as a yellow foam; ¹H NMR [(CD₃)₂SO] δ 8.94 (t, J=5.6 Hz, 1H), 8.75 (d, J=2.8 Hz, 1H), 8.34 (d, J=2.8 Hz, 1H), 4.28 (t, J=5.4 Hz, 2H), 4.02 (q, J=6.2 Hz, 2H), 3.74-3.43 (m, 8H), 3.13 (s, 3H), 1.43 (s, 18H). ¹³C NMR δ 265.6, 146.2, 145.3, 140.8, 135.6, 127.5, 122.4, 81.7, 67.5, 64.2, 54.3, 51.3, 41.4, 36.5, 29.5; identical to the sample in example 6.

### Example 13

### Table 1, Entry 7 and Scheme 10

### Preparation of tert-butyl 2-[N-(2-chloroethyl)-N-[2-[(methylsulfonyl)oxy]amino]-3,5-dinitrobenzoate (24)

A stirred solution of tert-butyl 2-chloro-3,5-dinitrobenzoate [Guenin, 1983] (22) 500 mg, 1.65 mmol) in DMF (1 mL) at 0° C. was treated with LiCl (70 mg, 1.65 mmol), followed dropwise by 1-aziridineethanol (0.33 mL, 4.12 mmol). The mixture was warmed to room temperature for 16 h, then diluted with saturated aqueous NaCl (40 mL) and refrigerated. The collected precipitate was purified by chromatography on silica gel, eluting with EtOAc/petroleum ether (3:4), followed by recrystallisation from EtOAc/petroleum ether to give tert-butyl 2-[N-(2-hydroxyethyl)-N-(2-chloroethyl)amino]-3,5-dinitrobenzoate (23) (407 mg, 63%) as a yellow solid: mp 106-107° C. ¹H NMR [(CD₃)₂SO] δ 8.73 (d, J=2.8 Hz, 1H), 8.44 (d, J=2.8 Hz, 1H), 4.58 (t, J=5.4 Hz, 1H), 3.75 (t, J=6.7 Hz, 2H), 3.55 (q, J=5.7 Hz, 2H), 3.46 (t, J=6.7 Hz, 2H), 3.19 (t, J=5.9 Hz, 2H), 1.60 (s, 9H). Anal. Calcd. for C₁₅H₂₀ClN₃O₇: C, 46.2; H, 5.2; N, 10.8; Cl, 9.1. Found: C, 46.5; H, 5.2; N, 10.7; Cl, 9.1%.

A stirred solution of 23 (640 mg, 1.64 mmol) in CH₂Cl₂ (10 mL) containing pyridine (0.34 mL, 4.28 mmol) was treated dropwise at 0° C. with a solution of (MsO)₂O (372 mg, 2.14 mmol) in CH₂Cl₂ (1.5 mL). The reaction mixture was allowed to warm to room temperature for 1 h, then treated with saturated aqueous NaHCO₃ (10 mL) and stirred for a further 0.5 h. The organic phase was washed with 1 N aqueous AcOH and water, then dried and evaporated under reduced pressure. Chromatography on silica gel, eluting with EtOAc/petroleum ether (1:1), followed by recrystallisation from EtOAc/petroleum ether gave ten-butyl 2-[N-(2-chloroethyl)-N-[(2-[(methylsulfonyl)oxy]ethyl]amino]-3,5-dinitrobenzoate (24) (686 mg, 89%) as a yellow solid: mp 95-96° C. ¹H NMR [(CD₃)₂SO] δ 8.80 (d, J=2.8 Hz, 1H), 8.52 (d, J=2.8 Hz, 1H), 4.29 (t, J=5.5 Hz, 2H), 3.73 (t, J=6.8 Hz, 2H), 3.49 (t, J=5.5 Hz, 2H), 3.45 (t, J=6.8 Hz, 2H), 3.12 (s, 3H), 1.60 (s, 9H). Anal. Calcd. for C₁₆H₂₂ClN₃O₉S: C, 41.1; H, 4.7; N, 9.0; Cl, 7.6. Found: C, 41.2; H, 4.8; N, 8.9; Cl, 7.8%.

### Further Processing of 24 to Give 2-[(2-chloroethyl)-2-[[(2-hydroxyethyl)amino]carbonyl]-4,6-dinitroanilino]ethyl methanesulfonate (31a)

A solution of 24 (646 mg, 1.38 mmol) in TFA (3 mL) was stirred at room temperature for 2 h, then concentrated to small volume (not to dryness) under reduced pressure. It was then partitioned between EtOAc and water, and the organic phase was dried and evaporated under reduced pressure. Trituration of the residue with iPr₂O and by recrystallisation of the resulting solid from EtOAc/hexane gave 2-[N-(2-chloroethyl)-N-[(2-[(methylsulfonyl)oxy]ethyl]amino]-3,5-dinitrobenzoic acid (38) (501 mg, 88%) as a yellow solid: mp 134-135° C. ¹H NMR [(CD₃)₂SO] δ 14.2 (v br, 1H), 8.81 (d, J=2.8 Hz, 1H), 8.60 (d, J=2.8 Hz, 1H), 4.28 (t, J=5.4 Hz, 2H), 3.71 (t, J=6.8 Hz, 2H), 3.53-3.42 (m, 4H), 3.12 (s, 3H). Anal. Calcd. for C₁₂H₁₄ClN₃O₉S: C, 35.0; H, 3.4; N, 10.2; Cl, 8.6. Found: C, 35.3; H, 3.5; N, 10.1; Cl, 8.9%.

A suspension of 38 (300 mg, 0.73 mmol) in CH₂Cl₂ (5 mL) was treated with oxalyl chloride (0.12 mL, 1.40 mmol) and DMF (one drop), and stirred at room temperature for 1.5 h. Evaporation of the volatiles under reduced pressure below 30° C., followed by azeotroping with benzene, gave the crude acid chloride. A solution of this in DMF (1 mL) was added dropwise to a stirred solution of 2-aminoethanol (6.7 mg, 1.10 mmol) and DIPEA (14.2 mg, 1.10 mmol) in dioxane/THF (1:1) (2 mL) at −5° C. The mixture was stirred at 0° C. for a further 5 min, then poured into 0.12 N aqueous MsOH (15 mL) and extracted with EtOAc (2×10 mL), The combined organic phase was washed with water, dried, and evaporated under reduced pressure. Chromatography on silica gel, eluting with EtOAc, followed by precipitation of the product from a CH₂Cl₂ solution with hexane, gave 2-[(2-chloroethyl)-2-[[(2-hydroxyethyl)amino]carbonyl]-4,6-dinitroanilino]ethyl methanesulfonate (31a) (272 mg, 82%) as a yellow gum, identical to a previous sample [PCT Int. Appl. WO 2005042471].

### Example 14

### Table 1, Entry 8 and Scheme 11

### Preparation of methyl 3-[(2-chloroethyl)-2-[(methylsulfonyloxy)ethyl]amino]-2,6-dinitrobenzoate (27a)

A solution of 3-chloro-2-nitrobenzoic acid (39) (20.0 g, 99.2 mmol) in H₂SO₂ (98%, 200 mL) was treated with Fuming nitric acid (12 mL) at room temperature, the reaction mixture was then stirred and heated at 140° C. for 2 h. The mixture was cooled and then poured into ice-water (600 mL), the white precipitate was collected, dried, gave 3-chloro-2,6-dinitrobenzoic acid (40) (21.5 g, 87%), the aqueous solution was extracted with EtOAc (3×200 mL), concentrated under reduced pressure and gave 3.0 g of product (12%, overall yield 99%). Acid 40 was methylated with MeOH/H₂SO₄ to give methyl 3-chloro-2,6-dinitrobenzoate (25) [Palmer et al., J. Med. Chem., 1992, 35, 3214].

A solution of ester 25 (1.15 g, 5.0 mmol) in dry DMF (10 mL) was treated with aziridineethanol (1.3 g, 15.0 mmol) and LiCl (0.5 g, 12 mmol) and stirred at room temperature for 6 h. The mixture was diluted with brine (40 mL) and extracted with EtOAc (2×60 mL). The combined organic fractions were washed with brine dried and concentrated under reduced pressure. Chromatography of the residue on silica gel, eluting with EtOAc/petroleum ether gave methyl 3-[(2-chloroethyl)(2-hydroxyethyl)amino]-2,6-dinitrobenzoate (26a) (0.96 g, 63% based on the consumed starting material) as a yellow oil: ¹H NMR (CDCl₃) δ 8.22 (d, J=9.4 Hz, 1H), 7.41 (d, J=9.4 Hz, 1H), 3.98 (s, 3H), 3.77 (vbr s, 1H), 3.58 (m, 8H); ¹³C NMR δ 163.1, 148.5, 147.9, 127.9, 123.2, 122.2, 59.7, 54.2, 53.9, 41.0. HRMS (FAB) calcd for C₁₂H₁₄³⁵ClN₃O₇ [M]⁺ m/z 347.0520; found 347.0521.

Further elution gave starting material (0.16 g).

A stirred solution of 26a (0.39 g, 1.12 mmol) in CH₂Cl₂ (15.0 mL) was cooled at 0° C. and then treated with Et₃N (0.4 mL) and MsCl (0.2 mL, 2.8 mmol) for 1.5 h. The mixture was diluted with water and extracted with EtOAc (2×50 mL). The combined organic fractions were washed with water (3×), dried, and concentrated under reduced pressure. Chromatography on silica gel and elution with EtOAc/petroleum ether (9:1) gave mesylate 27a (0.38 g, 80%) as a yellow oil: ¹H NMR [(CD₃)₂SO] δ 8.31 (d, J=9.5 Hz, 1H), 7.74 (d, J=9.5 Hz, 1H), 4.31 (t, J=5.2 Hz, 2H), 3.88 (s, 3H), 3.78 (t, J=6.3 Hz, 2H), 3.70 (t, J=5.2 Hz, 2H), 3.64 (t, J=5.9 Hz, 2H), 3.14 (s, 3H); ¹³C NMR δ 163.0, 147.5, 138.1, 136.2, 128.3, 125.9, 123.6, 66.7, 54.8, 53.9, 49.8, 36.6. HRMS (FAB) calcd for C₁₃H₁₇³⁵ClN₃O₉S [M+H]⁺ m/z 426.0374; found 426.0372.

### Example 15

### Table 1, Entry 8 and Scheme 11

### Preparation of methyl 3-[(2-bromoethyl)-2-[(methylsulfonyloxy)ethyl]amino]-2,6-dinitrobenzoate (27b)

A solution of methyl 3-chloro-2,6-dinitrobenzoate (25) (1.15 g, 5.0 mmol) in dry DMF (10 mL) was treated with aziridineethanol (1.3 g, 15.0 mmol) and LiBr (4.35 g, 50 mmol) and stirred at room temperature for 6 h. The mixture was diluted with brine (40 mL) and extracted with EtOAc (2×60 mL). The combined organic fractions were washed with brine dried and concentrated under reduced pressure. Chromatography of the residue on silica gel, eluting with EtOAc/petroleum ether gave methyl 3-[(2-chloroethyl)(2-hydroxyethyl)amino]-2,6-dinitrobenzoate (26b) (0.40 g, 36% based on consumed starting material) as a yellow oil: ¹H NMR [(CD₃)₂SO] δ 8.24 (d, J=9.4 Hz, 1H), 7.62 (d, J=9.4 Hz, 1H), 4.79 (vbr s, 1H), 3.78 (s, 3H), 3.77 (t, J=6.2 Hz, 2H), 3.65 (t, J=6.1 Hz, 2H), 3.53 (t, J=5.4 Hz, 2H), 3.36 (t, J=5.3 Hz, 2H); ¹³C NMR δ 163.2, 147.6, 136.4, 134.5, 128.1, 126.3, 121.9, 58.1, 53.8, 53.6, 52.5, 29.7. 38b: HRMS (FAB) calcd for C₁₂H₁₅⁷⁹BrN₃O₇ [M+H]⁺ m/z 392.0093; found 392.0093.

Later eluates gave starting material (0.50 g).

A stirred solution of 26b (0.14 g, 0.36 mmol) in CH₂Cl₂ (15.0 mL) was cooled at 0° C. and then treated with Et₃N (0.2 mL) and MsCl (0.1 mL, 1.4 mmol) for 1.5 h. The mixture was diluted with water and extracted with EtOAc (2×50 mL). The combined organic fractions were washed with water (3×), dried, and concentrated under reduced pressure. Chromatography on silica gel and elution with EtOAc/petroleum ether (9:1) gave mesylate 27b (0.13 g, 77%) as a yellow oil: ¹H NMR [(CD₃)₂SO] δ 8.31 (d, J=9.5 Hz, 1H), 7.74 (d, J=9.5 Hz, 1H), 4.31 (t, J=5.2 Hz, 2H), 3.88 (s, 3H), 3.65 (m, 6H), 3.14 (s, 3H); ¹³C NMR δ 162.9, 147.3, 138.1, 136.3, 128.3, 125.9, 123.5, 66.7, 53.9, 52.7, 49.7, 36.6, 29.9. HRMS (FAB) calcd for C₁₃H₁₇⁷⁹BrN₃O₉S [M+H]⁺ m/z 469.9869; found 469.9865.

### Example 16

### Table 1, entry 8 and Scheme 11

### Preparation of methyl 3-[N-(2-iodoethyl)-N-[2-[(methylsulfonyl)oxy]ethyl]amino]-2,6-dinitrobenzoate (27c)

A stirred solution of methyl 3-chloro-2,6-dinitrobenzoate (25) (140 mg, 0.54 mmol) in DMF (1.5 mL) at room temperature was treated with NaI (405 mg, 2.7 mmol) for 5 min, then cooled to 0° C. and treated with aziridineethanol (0.15 mL, 1.87 mmol). The mixture was stirred at 30° C. for 16 h, then diluted with 1N aqueous AcOH (15 mL) and extracted with EtOAc (2×15 mL). The combined organic phases were washed with water (2×), dried, and concentrated under reduced pressure. The residue was chromatographed on silica gel, eluting with EtOAc/petroleum ether (1:1). The middle fractions were combined and concentrated to small volume, then hexane was added to precipitate methyl 3-[N-(2-hydroxyethyl)-N-2-(iodoethyl)amino]-2,6-dinitrobenzoate (26c) (138 mg, 58%) as a yellow gum: ¹H NMR [(CD₃)₂SO] δ 8.24 (d, J=9.7 Hz, 1H), 7.59 (d, J=9.7 Hz, 1H), 4.79 (t, J=5.2 Hz, 1H), 3.87 (s, 3H), 3.71 (t, J=7.2 Hz, 2H), 3.53 (q, J=5.1 Hz, 2H), 3.40-3.33 (m, 4H). HRMS (FAB) calcd. for C₁₂H₁₅IN₃O₇ [MH⁺] m/z 439.9955; found 439.9960.

A stirred solution of 26c (126 mg, 0.29 mmol) in CH₂Cl₂ (4 mL) at 0° C. was treated with pyridine (60 uL, 0.75 mmol), followed by the slow addition of a solution of methanesulfonic anhydride (65 mg, 0.37 mmol) in CH₂Cl₂ (1 mL). The reaction mixture was warmed to room temperature for 30 min, then treated with satd. aqueous NaHCO₃ (5 mL) and stirred for a further 30 min. The organic phase was washed with 1N aqueous AcOH, water (2×), dried, and concentrated under reduced pressure. The residue was chromatographed on silica gel, eluting with EtOAc/petroleum ether (2:1), followed by precipitation of the product from CH₂Cl₂ solution with hexane, to give methyl 3-[N-(2-iodoethyl)-N-[2-[(methylsulfonyl)oxy]ethyl]amino]-2,6-dinitrobenzoate (27c) (137 mg, 92%) as a yellow gum: ¹H NMR [(CD₃)₂SO] δ 8.30 (d, J=9.6 Hz, 1H), 7.72 (d, J=9.6 Hz, 1H), 4.30 (t, J=5.2 Hz, 2H), 3.88 (s, 3H), 3.71 (t, J=5.2 Hz, 2H), 3.62 (t, J=7.1 Hz, 2H), 3.36 (t, J=7.1 Hz, 2H), 3.14 (s, 3H). HRMS (FAB) calcd. for C₁₃H₁₇IN3O₉S [MH⁺] m/z 517.9730; found 517.9734.

### Example 17

### Table 1, Entry 9 and Scheme 12

### Preparation of tert-butyl 3-[N-(2-chloroethyl)-N-[2-[(methylsulfonyl)oxy]ethyl]amino]-2,6-dinitrobenzoate (30)

A stirred solution of t-Bu ester (28) [Guenin & Schneider, Helv. Chim. Acta, 1983, 66, 1101] (200 mg, 0.66 mmol) in DMF (0.8 mL) at 0° C. was treated with LiCl (28 mg, 0.66 mmol), followed by aziridineethanol (144 mg, 1.65 mmol). The mixture was stirred at 45° C. for 16 h, then diluted with 1N aqueous AcOH (15 mL) and extracted with EtOAc (2×15 mL). The combined organic phases were washed with water (2×), dried, and concentrated under reduced pressure. The residue was purified by chromatography on silica gel, eluting with EtOAc/petroleum ether (1:1), followed by precipitation of the product from CH₂Cl₂ solution with hexane, to give tert-butyl 3-[N-(2-chloroethyl)-N-(2-hydroxyethyl)amino]-2,6-dinitrobenzoate (29) (131 mg, 51%) as a yellow gum: ¹H NMR [(CD₃)₂SO] δ 8.20 (d, J=9.6 Hz, 1H), 7.58 (d, J=9.6 Hz, 1H), 4.78 (t, J=5.2 Hz, 1H), 3.81-3.74 (m, 2H), 3.74-3.66 (m, 2H), 3.53 (q, J=5.3 Hz, 2H), 3.34 (t, J=5.5 Hz, 2H), 1.50 (s, 9H). HRMS (FAB) calcd. for C₁₅H₂₁³⁵ClN₃O₇ [MH⁺] m/z 390.1068; found 390.1063.

A stirred solution of 29 (139 mg, 0.36 mmol) in CH₂Cl₂ (4 mL) at 0° C. was treated with pyridine (75 uL, 0.93 mmol), followed by the dropwise addition of a solution of methanesulfonic anhydride (81 mg, 0.46 mmol) in CH₂Cl₂ (1 mL). The reaction mixture was warmed to room temperature for 1 h, then treated with satd. aqueous NaHCO₃ (5 mL) and stirred for a further 30 min. The organic phase was washed with 1N aqueous AcOH, water (2×), dried, and concentrated under reduced pressure. The residue was chromatographed on silica gel, eluting with EtOAc/petroleum ether (1:1), followed by precipitation of the product from CH₂Cl₂ solution with hexane, to give tert-butyl 3-[N-(2-chloroethyl)-N-[2-[(methylsulfonyl)oxy]ethyl]amino]-2,6-dinitrobenzoate (30) (147 mg, 88%) as a yellow gum: ¹H NMR. [(CD₃)₂SO] δ 8.26 (d, J=9.5 Hz, 1H), 7.70 (d, J=9.5 Hz, 1H), 4.30 (t, J=5.2 Hz, 2H), 3.76 (t, J=6.2 Hz, 2H), 3.68 (t, J=5.2 Hz, 2H), 3.62 (t, J=6.2 Hz, 2H), 3.15 (s, 3H), 1.50 (s, 9H). HRMS (FAB) calcd. for C₁₆H₂₃³⁵ClN₃O₉S [MH⁺] m/z 468.0844; found 468.0829.

### Example 18

### Scheme 13

### Preparation of 2-{[2-((2-bromoethyl){2-[(methylsulfonyl)oxy]ethyl}amino)-3,5-dinitrobenzoyl]amino}ethyl 2-amino-4-methylpentanoate (44)

Reaction of 37 by the method of Scheme 13 gave 2-{[2-((2-bromoethyl){2-[(methylsulfonyl)oxy]ethyl}amino)-3,5-dinitrobenzoyl]amino}ethyl 2-[(tert-butoxycarbonyl)amino]-4-methylpentanoate (43), as a yellow foam: ¹H NMR [(CD₃)₂SO] δ 8.61 (d, J=2.8 Hz, 1H), 8.52 (d, J=2.8 Hz, 1H), 7.52 (vbs, 1H), 4.88 (d, J=6.6 Hz, 1H), 4.47 (m, 1H), 4.37 (t, J=5.3 Hz, 1H), 4.35 (m, 2H), 4.15 (m, 1H), 3.75 (m, 2H), 3.62-3.48 (m, 6H), 3.02 (s, 3H), 1.65-1.48 (m, 3H), 1.32 (s, 9H), 0.95 (t, J=6.9 Hz, 6H); ¹³C NMR δ 173.6, 165.5, 156.0, 146.2, 145.8, 142.0, 136.2, 128.4, 122.7, 80.4, 67.2, 63.1, 55.5, 52.7, 52.0, 40.7, 39.4, 37.5, 28.7, 28.1, 24.9, 22.7, 21.8; HRMS (FAB) calcd for C₂₅H₃₉⁷⁹BrN₅O₁₂S [M+H]⁺ m/z 712.1499; found 712.1485.

Acid deblocking of 43 with trifluoroacetic acid gave 44 as a yellow foam: ¹H NMR [(CD₃)₂SO] δ 8.96 (t, J=5.6 Hz, 1H), 8.77 (d, J=2.8 Hz, 1H), 8.40 (vbs, 1H), 8.33 (d, J=2.8 Hz, 1H), 4.40-4.25 (m, 4H), 4.0 (vbr, 1H), 3.62-3.55 (m, 4H), 3.51-3.43 (m, 4H), 3.14 (s, 3H), 1.83-1.58 (m, 3H), 0.89 (m, 6H); ¹³C NMR δ 169.8, 165.5, 145.9, 145.4, 140.9, 135.5, 131.1, 127.4, 126.0, 122.5, 67.5, 63.8, 54.3, 51.2, 50.5, 38.2, 36.5, 29.7, 23.6, 22.0, 21.7; HRMS (FAB) calcd for C₂₀H₃₀⁷⁹BrN₅O₁₀S [M+H]⁺ m/z 612.0975; found 612.0975.

### Example 19

### Scheme 14

### Preparation of 2-[(2-bromoethyl)-2-[[[2-(beta-D-gulopyranosyloxy)ethyl]amino]carbonyl]-4,6-dinitroanilino]ethyl methanesulfonate (48)

Reaction of 37 as in Scheme 14, and chromatography on silica gel, eluting with EtOAc/petroleum ether (2:1) gave a product that was precipitated from a CH₂Cl₂ solution with i-Pr₂O to give 2-[(2-bromoethyl)-2,4-dinitro-6-[[[2,3,4,6-tetra-O-acetyl-beta-D-gulopyranosyl)oxy]ethyl]amino]carbonyl]anilino]ethyl methanesulfonate (47) (2.04 g, 82%) as a yellow solid: mp 70-73° C.; ¹H NMR [(CD₃)₂SO] δ 8.84 (t, J=5.5 Hz, 1H), 8.75 (d, J=2.8 Hz, 1H), 8.31 (d, J=2.8 Hz, 1H), 5.27 (d, J=3.2 Hz, 1H), 5.17 (dd, J=10.4, 3.5 Hz, 1H), 4.99 (dd, J=10.3, 8.0 Hz, 1H), 4.78 (d, J=8.0 Hz, 1H), 4.28 (t, J=5.4 Hz, 1H), 4.22 (t, J=6.5 Hz, 1H), 4.11-3.99 (m, 2H), 3.94-3.84 (m, 1H), 3.77-3.68 (m, 1H), 3.63-3.37 (m, 8H), 3.13 (s, 3H), 2.13 (s, 3H), 2.01 (s, 3H), 1.97 (s, 3H), 1.92 (s, 3H. ¹³C NMR [(CD₃)₂SO] δ 169.8, 169.7, 169.4, 169.1, 165.4, 145.8, 145.3, 140.8, 135.8, 127.4, 122.3, 100.1, 70.2, 69.9, 68.5, 67.4, 67.3 (2), 61.2, 54.3, 51.1, 39.4, 36.4, 29.7, 20.4, 20.3, 20.2 (2).

An ice-cold solution of LiOH (0.05 in MeOH/water/THF (3:1:1) (276 mL, 13.8 mmol) was added to 47 (1.91 g, 2.30 mmol), and the mixture was stirred at 0° C. for 3 h. After being quenched with AcOH (0.8 mL) the solution was concentrated under reduced pressure to 50 mL, extracted with EtOAc (3×50 mL) and the combined organic phases were dried and concentrated under reduced pressure. The residue was chromatographed on silica gel, eluting with EtOAc/MeOH (9:1), and the eluate was concentrated to small volume and diluted with hexane. The resulting precipitate was stirred as a suspension in i-Pr₂O and re-collected to give 48 (1.17 g, 77%) as a yellow solid: mp (indefinite); ¹H NMR [(CD₃)₂O] δ 8.77-8.69 (m, 2H), 8.33 (d, J=2.8 Hz, 1H), 4.76 (d, J=3.9 Hz, 1H), 4.69 (d, J=5.2 Hz, 1H), 4.51 (t, J=5.6 Hz, 1H), 4.33 (d, J=4.4 Hz, 1H), 4.28 (t, J=5.4 Hz, 2H), 4.15 (d, J=7.1 Hz, 1H), 3.93-3.85 (m, 1H), 3.70-3.43 (m, 12H_{—}, 3.38-3.26 (m, 3H), 3.13 (s, 3H); ¹³C NMR [(CD₃)₂O] δ 165.3, 145.8, 140.9, 136.0, 127.5, 122.2, 103.5, 75.2, 73.2, 70.5, 68.0, 67.5, 67.0, 60.3, 54.3, 51.1, 39.6, 36.5, 29.8.

### Example 20

### Scheme 15

### Preparation of 2-((2-bromoethyl)-2-{11-[2-((2-bromoethyl){2-[(methylsulfonyl)oxy]ethyl}amino)-3,5-dinitrophenyl]-6-hydroxy-6-oxido-11-oxo-5,7-dioxa-2,10-diaza-6-phosphaundec-1-anoyl}-4,6-dinitroanilino)ethyl methanesulfonate (52)

Reaction of 37 as in Scheme 15 gave 2-((2-bromoethyl)-2-{11-[2-((2-bromoethyl){2-[(methylsulfonyl)oxy]ethyl}amino)-3,5-dinitrophenyl]-6-oxido-11-oxo-5,7-diaza-6-phosphaundec-1-anoyl}-4,6-dinitroanilino)ethyl methanesulfonate (51) as a yellow oil: ¹H NMR [(CD₃)₂SO] δ 8.99 (t, J=5.6 Hz, 2H), 8.75 (d, J=2.8 Hz, 2H), 8.40 (d, J=2.8 Hz, 2H), 6.98 (d, J=709 Hz, 1H), 4.28 (t, J=5.5 Hz, 4H), 4.20 (m, 4H), 3.58 (m, 8H), 3.48 (m, 8H), 3.13 (s, 6H); ¹³C NMR δ 165.5, 145.9, 145.3, 140.9, 135.6, 127.6, 122.4, 67.4, 63.4, 54.3, 51.1, 39.7, 36.4, 29.7; HRMS (FAB) calcd for C₂₈H₃₈⁷⁹Br₂N₈O₁₉PS₂ [M+H]⁺ m/z 1042.9799; found 1042.9786.

A solution of 51 (80 mg) in a solution of CCl₄/H₂O/NMM/Py/CH₃CN (2.5/1.0/1.0/6.0/1.0) (5 mL) stirred at room temperature for 5 min., poured to ice-HCl (1N), extracted with CH₂Cl₂ (2×50 mL). The combined organic phase were washed with brine, dried, concentrated under reduced pressure gave 52 (66 mg, 83%) as a yellow foam: ¹H NMR [(CD₃)₂SO] δ 8.98 (t, J=5.6 Hz, 2H), 8.74 (d, J=2.8 Hz, 2H), 8.38 (d, J=2.8 Hz, 2H), 4.28 (t, J=5.4 Hz, 4H), 4.06 (m, 4H), 3.65 (vbr, 1H), 3.58 (m, 8H), 3.48 (m, 8H), 3.13 (s, 6H); ¹³C NMR δ 165.6, 146.0, 145.2, 140.8, 135.5, 127.7, 122.5, 67.5, 64.1, 54.9, 54.2, 51.0, 36.4, 29.8; HRMS (FAB) calcd for C₂₈H₃₈⁷⁹Br₂N₈O₂₀PS₂[M+H]⁺ m/z 1058.9748; found 1058.9755.

### Example 21

### Scheme 16

### Preparation of 2-[(2-bromoethyl)-2,4-dinitro-6-[[[2-(phosphonooxy)ethyl]amino]-carbonyl]anilino]ethyl methanesulfonate (33b)

A solution of 1 (10 g, 34 mmol) in toluene (200 DMF (0.45 mL), and (COCl)₂ (22 g, 3 3 equiv) was heated to reflux for 1 h, then cooled and evaporated to dryness under reduced pressure and azeotroped once with toluene to remove excess (COCl)₂. The residue was dissolved in toluene (100 mL), cooled in an ice-bath, and treated with 2-aminoethanol (6.6 g, 2.5 equiv). The mixture was stirred for 30 min, then acidified with 1N HCl to pH 4-5, washed with water, and evaporated under reduced pressure to give 2-bromo-3,5-dinitro-N-(2-hydroxyethyl)benzamide (2) as a white solid (8.1 g, 70% yield) which was used directly.

A solution of 2 (49 g, 145 mmol) in DCM (500 mL) mL was cooled in an ice-bath, and 3,4-dihydro-2H-pyran (2.5 equiv) and p-toluenesulfonic acid (0.5 g) were added. The reaction mixture was stirred for 2 h, then concentrated under reduced pressure to give 2-bromo-3,5-dinitro-N-[2-(tetrahydro-2H-pyran-2-yloxy)ethyl]benzamide (3) (53.4 g, 86% yield) as an oil that was used directly.

A solution of 3 (10 g, 30 mmol) in THF (100 mL) was treated with aziridineethanol (6 g, 2.5 equiv) and NaBr (0.5 g) 1 equiv) for 44 h. The mixture was then partitioned between water and DCM, and the organic layer was evaporated under reduced pressure to give 2-[(2-bromoethyl)(2-hydroxyethyl)amino]-3,5-dinitro-N-[2-(tetrahydro-2H-pyran-2-yloxy)ethyl]benzamide (4) (9.8 g, 81% yield, HPLC purity 84%) as a yellow solid: mp 121-123° C.; NMR as above.

A solution of 4 (9.8 g, 24 mmol) in DCM (100 mL) and pyridine (4 g, 2.6 equiv) was treated dropwise with methanesulfonic anhydride (3.7 g, 1.1 equiv) at 5° C., and the mixture was then allowed rise to room temperature for 16 h. MeOH (10 mL) was added, and the mixture was stirred for 15 min at room temperature to remove excess anhydride. Methanesulfonic acid was then added (3.84 g, 40 mmol, 2.06 equiv) was added, and the mixture was stirred for 2 h, when HPLC showed complete conversion to 5. The mixture was extracted with water (2×50 mL), dried (anhydrous MgSO₄), filtered, and concentrated to small volume (not to dryness) under reduced pressure. The residue was diluted with DCM (50 mL) and then re-concentrated (2×) to remove traces of MeOH, then dried under vacuum to give 5 as a yellow foam (8.2 g, 85% yield; HPLC purity 92.5%); NMR spectra as above.

A solution of 5 (1.0 g, 2.0 mmol) in DCM (10 mL) was cooled externally to −5° C. and treated sequentially with di-tert-butyl diisopropylphosphoramidite (0.8 mL, 1.2 equiv), followed by dicyanoimidazole (0.28 g, 1.2 equiv). The mixture was then diluted with DCM (10 mL) and treated with peracetic acid (2 equivs, 35 wt %) and stirred for a further 2 h at 20° C., then washed twice with dilute aqueous sodium thiosulfate, followed by water. The organic layer was then concentrated to small volume (ca. 5 mL) and treated with methanesulfonic acid (6 equiv based on 5) for two hours. The resultant oil was separated by decantation of the mother liquor, and was then diluted with EtOAc (10 mL). The resulting solution was filtered, then seeded with crystalline 33b, and after 20 h at 20° C. the solid was collected by filtration to give 33b (0.55 g, 48% yield, HPLC purity 96.4%). NMR as above.

It will be apparent to those skilled in the art that the processes of the present invention could be readily applied without undue experimentation and with appropriate selection of starting material, that other examples of compounds of formulae (II, III and IV) could be expeditiously prepared.

It is an advantage of the processes of the present invention that the processes described above of Scheme 1 provide significantly higher-yielding and more convenient synthetic routes to the general class of asymmetric (hetero)aromatic mustards.

### Applications of the Compounds Obtained by the Processes of the Present Invention

The compounds of formula (IV), and the compounds of formula (II) where Z is —NHR² and Q is —OH or —OP(O)(OH)₂, obtained from the processes of the present invention can be used in a method of treatment of the human or animal body. Such treatment includes a method of treating the growth of neoplastic cells in a patient with neoplastic disease which comprises administering to a patient in need of treatment compounds of formula (IV) or (II) of the invention, or where the compounds of formula (IV) or (II) obtained are suitable as substrates as part of an ADEPT or GDEPT therapy system. Neoplastic diseases include leukaemia and solid tumours such as breast, bowel and lung tumours including small cell lung carcinoma.

It will be understood that where treatment of tumours is concerned, treatment includes any measure taken by the physician to alleviate the effect of the tumour on a patient. Thus, although complete remission of the tumour is a desirable goal, effective treatment will also include any measures capable of achieving partial remission of the tumour as well as a slowing down in the rate of growth of a tumour including metastases. Such measures can be effective in prolonging and/or enhancing the quality of life and relieving the symptoms of the disease.

Compounds of formula (IV) and (II) obtained by the processes of the present invention may be used in a method of treatment of neoplastic disease in a patient, which method comprises administering to a patient in need of treatment an effective amount of a compound of formula (IV) or (II). The compounds obtained may be administered in the form of a pharmaceutical composition.

A number of the compounds described herein also have utility as intermediates, for preparing compounds of the formula (IV).

Where in the foregoing description reference has been made to reagents or other integers having known equivalents, then those equivalents are incorporated herein as if individually set forth.

While this invention has been described with reference to certain embodiments and examples, it is to be appreciated that modifications and variations may be made to embodiments and examples without departing from the scope of the invention as defined in the following claims.

1. A method of preparing a compound of formula (II) 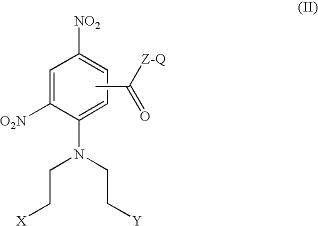
wherein:
Z represents —OR¹ or —N(R²)R^{2a}—,
where R¹ is lower alkylene (C₁-C₆), R² is lower alkyl or H and R^{2a} is lower alkylene (C₁-C₆) or H;
Q is absent when R^{2a} is H and is otherwise selected from the group consisting of H, —OH and protected forms of —OH;
one of X and Y is halogen and the other is —OSO₂R³,
where R³ is selected from the group consisting of lower alkyl (C₁-C₆), phenyl and CH₂phenyl;
the method comprising the steps of:
(a) reacting a compound of formula (I) 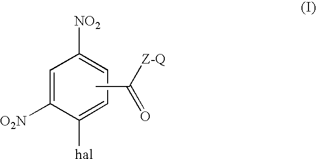
wherein hal is a halogen atom, Z is as defined above for formula (II), and Q is absent when R^{2a} is H and is otherwise selected from the group consisting of H and protected forms of OH,
with aziridineethanol or a 2-[(2-haloethyl)amino]ethanol in the presence of a metal halide, to form a compound of the formula (III): 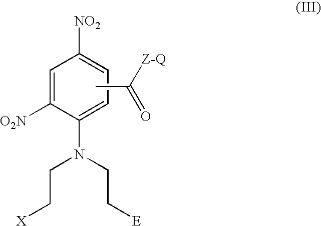
wherein one of X and E is halogen and the other is hydroxy, and Z and Q are as defined above, and
(b) reacting the compound of formula (III) so obtained with an alkyl- or arylsulfonyl halide or alkyl- or arylsulfonyl anhydride to obtain a compound of the formula (II).

2. A method as claimed in claim 1, wherein the compound of formula (I) is reacted with aziridineethanol.

3. A method as claimed in claim 1, wherein the compound of formula (I) is reacted with a 2-[(2-haloethyl)amino]ethanol.

4. A method as claimed claim 1 wherein the metal halide comprises a lithium halide.

5. A method as claimed claim 1, wherein step (a) comprises preparing a compound of formula (IIa) from a compound of formula (Ia): 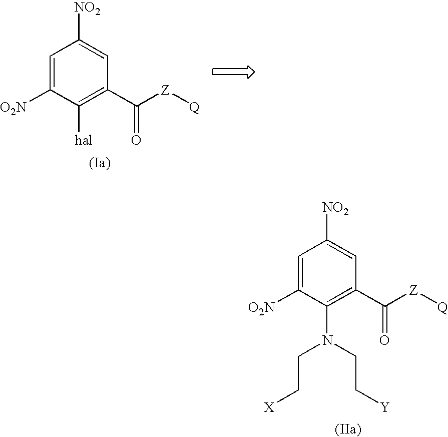
wherein:
Z-Q in formulae (Ia) and (IIa) represents OtBu or NH(CH₂)ₙOJ, where n is 2 or 3 and J is THP (tetrahydropyranyl), P(O)(OtBu)₂, methyl tetraacetyl-β-glucuronide or COK, where K is a protected mono-, di- or tripeptide;
hal in formula (Ia) represents Cl, Br or I; and
one of X and Y in formula (IIa) represents halogen and the other represents —OSO₂R³, where R³ is lower alkyl (C1-C6).

6. A method as claimed in claim 5, wherein:
Z-Q in formulae (Ia) and (IIa) represents OtBu or NH(CH₂)ₙOJ, where n is 2 or 3 and J is THP (tetrahydropyranyl), P(O)(OtBu)₂, or methyl tetraacetyl-β-glucuronide;
hal in formula (Ia) represents Cl or Br; and
one of X and Y in formula (IIa) represents halogen and the other represents —OSO₂Me.

7. A method as claimed in claim 5, wherein:
Z-Q in formulae (Ia) and (IIa) represents OtBu or NH(CH₂)₂OJ, where J is THP (tetrahydropyranyl) or P(O)(OtBu)₂;
hal in formula (Ia) represents Cl or Br; and
X and Y in formula (IIa) separately represent Br and OSO₂Me.

8. A method according to claim 1 of preparing a compound of the formula: 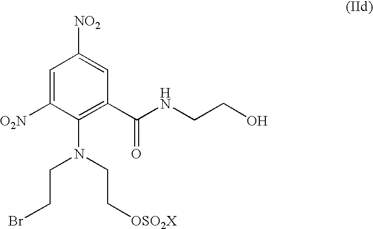
wherein X is alkyl or aryl;
wherein the method comprises the following steps:
(a) reacting a compound of the general formula (I) as defined in claim 1 having the following formula: 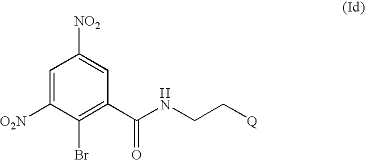
wherein Q is a protected form of —OH, with aziridineethanol or a 2-[(2-haloethyl)amino]ethanol in the presence of a metal halide, to form a compound of the general formula (III) as defined in claim 1 having the following formula: 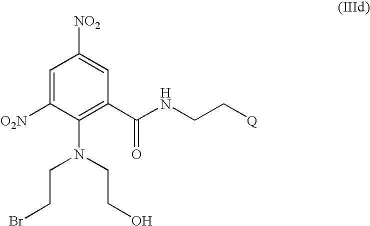
Wherein Q is as defined above; and
(b) reacting the compound of formula (IIId) with an alkyl- or arylsulfonyl halide or alkyl- or arylsulfonyl anhydride to form a compound of the formula (IId) as defined above.

9. A method as claimed in claim 8, wherein the compound of formula (Id) is reacted with aziridineethanol.

10. A method as claimed in claim 8, wherein the metal halide comprises sodium bromide.

11. A method as claimed in claim 8, wherein the alkyl- or arylsulfonyl halide or anhydride is methansulfonic anhydride, and the compound of formula (IIb) prepared is

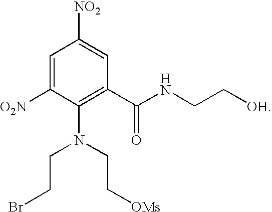

12. A method as claimed in claim 8, wherein the compound of formula (Id) is prepared by reacting a compound of the formula: 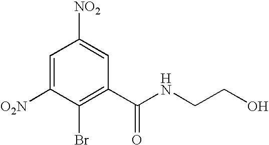
with dihydropyran, to form a compound of formula (Id) in which Q is THP.

13. A method as claimed in claim 9, wherein the method includes the additional step of further processing the compound of formula (IId) to form a compound of the following formula:

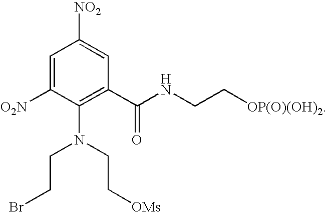

14. A method as claimed in claim 13, wherein the further processing comprises reacting the compound of formula (IId) with iPr₂NP(OtBu), to form a phosphate ester of the compound of formula (IId), followed by deprotecting the phosphate ester.

15. A method as claimed in claim 14, wherein the phosphate ester is deprotected by reaction with MsOH.

16. A method of preparing a compound of formula (III) as defined in claim 1, the method comprising the step of reacting a compound of formula (I) as defined in claim 1 with aziridineethanol or a 2[(2-haloethyl)amino]ethanol in the presence of a metal halide.

17. A method of preparing a compound of formula (IV) 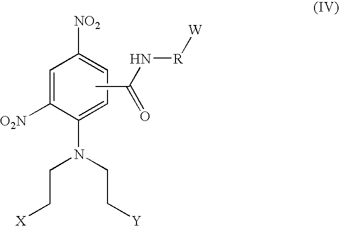
where W is —OH or —OP(O)(OH)₂;
R is lower alkylene (C₁-C₆);
and one of X and Y is halogen and the other is —OSO₂R³; wherein R³ is selected from the group consisting of lower alkyl (C₁-C₆), phenyl and CH₂phenyl;
the method comprising the steps of:
(a) reacting a compound of formula (I) as defined in claim 1 with aziridineethanol or a 2-[(2-haloethyl)amino]ethanol in the presence of a metal halide to obtain a compound of formula (III) as defined in claim 1;
(b) reacting the compound of formula (III) so obtained with an alkyl- or aryl sulfonyl halide or alkyl- or arylsulfonyl anhydride to obtain a compound of formula (II) as defined in claim 1; and
(c) optionally, carrying out further processing of the compound of formula (II) as required to obtain a compound of formula (IV).

18. A method as claimed in claim 17, wherein the compound of formula (I) is reacted with aziridineethanol.

19. A method as claimed in claim 17, wherein the compound of formula (I) is reacted with a 2-[(2-haloethyl)amino]ethanol.

20. A method as claimed in claim 17, wherein the metal halide comprises lithium halide.

21. A method as claimed in claim 17, wherein the compound of formula (IV) prepared is selected from the group consisting of:
2-((2-chloroethyl)-2-{[(2-hydroxyethyl)amino]carbonyl}-4,6-dinitroanilino)ethyl methanesulfonate;
2-((2-bromoethyl)-2-{[(2-hydroxyethyl)amino]carbonyl}-4,6-dinitroanilino)ethyl methanesulfonate;
2-((2-iodoethyl)-2-{[(2-hydroxyethyl)amino]carbonyl}-4,6-dinitroanilino)ethyl methanesulfonate;
2-((2-bromoethyl)-2-{[(2-hydroxyethyl)amino]carbonyl}-4,6-dinitroanilino)ethyl 1-butanesulfonate;
2-[(2-bromoethyl)-2,4-dinitro-6-[[[2-(phosphonooxy)ethyl]amino]-carbonyl]anilino]ethyl methanesulfonate;
2-[(2-bromoethyl)-2,4-dinitro-6-({[2-(phosphonooxy)ethyl]amino}carbonyl)anilino]ethyl 1-butanesulfonate;
2-[(2-chloroethyl)-2,4-dinitro-6-[[[2-(phosphonooxy)ethyl]amino]-carbonyl]anilino]ethyl methanesulfonate; and
2-[(2-iodoethyl)-2,4-dinitro-6-({[2-(phosphonooxy)ethyl]amino}carbonyl)-anilino]ethyl methanesulfonate.

22. A method as claimed in claim 17, wherein the compound of formula (IV) prepared is 2-[(2-bromoethyl)-2,4-dinitro-6-[[[2-(phosphonooxy)ethyl]amino]-carbonyl]anilino]ethyl methanesulfonate.

23. A method of preparing a compound of formula (IV) as defined in claim 17, characterised in that the process includes the step of reacting a compound of formula (I) as defined in claim 1 with aziridineethanol or a 2-[(2-haloethyl)amino]ethanol in the presence of a metal halide to obtain a compound of formula (III) as defined in claim 1.

24. A method of preparing a compound of formula (II) as defined in claim 1, characterised in that the process includes the step of reacting a compound of formula (I) as defined in claim 1 with aziridineethanol or a 2-[(2-haloethyl)amino]ethanol in the presence of a metal halide to obtain a compound of formula (III) as defined in claim 1.

25. A compound of formula (IIb): 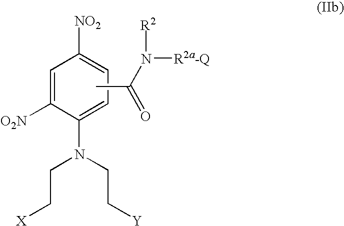
wherein:
one of X and Y is halogen and the other is —OSO₂R³, where R³ is selected from the group consisting of lower alkyl C₁-C₆), phenyl and CH₂phenyl;
R² represents lower alkyl (C₁-C₆), or H, and R^{2a} represents lower alkylene (C₁-C₆);
Q is selected from the group consisting of:
(1) —OR⁴, where R⁴ is a mono-, di- or tripeptide,
(2) —OR⁶, where R ^{6̅} is a mono-, di- or trisaccharide,
(3) —O(C═O)K, where K is (a) lower alkyl (C₁-C₆) optionally substituted with one or more groups selected from OH, NH₂, NHR⁵ and NR⁵R^{5a}, where each R⁵ and R^{5a} is independently lower alkyl (C₁-C₃), or R⁵R^{5a} taken together represents pyrrolyl, piperidinyl, piperazinyl, 4-methylpiperazinyl, N-imidazolyl or 2-, 3- or 4-pyridyl, or K is (b) (CH₂)ₙCONH(CH₂)ₙNR⁵R^{5a}, where n is 1, 2 or 3, and R⁵ and R^{5a} are as defined above, and 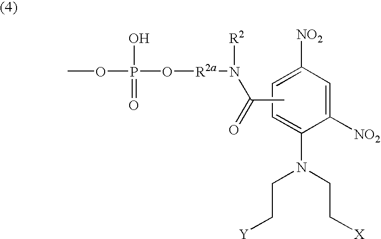
wherein R², R^{2a}, X and Y are as defined immediately above;
and wherein, when R² is lower (C₁-C₆) alkyl, Q can also represent —OP(O)(OH)₂.

26. A compound of formula (V): 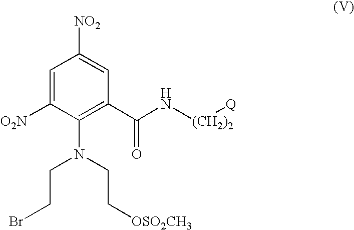
wherein
Q is selected from the group consisting of:
(1) —OR⁴, where R⁴ is a mono-, di- or tripeptide,
(2) —OR⁶, where R⁶ is a mono-, di- or trisaccharide,
(3) —O(C═O)K, where K is (a) lower alkyl (C₁-C₆) optionally substituted with one or more groups selected from OH, NH₂, NHR⁵ and NR⁵R^{5a}, where each R⁵ and R^{5a} is independently lower alkyl (C₁-C₃), or R⁵R^{5a} taken together represents pyrrolyl, piperidinyl, piperazinyl, 4-methylpiperazinyl, N-imidazolyl or 2-, 3- or 4-pyridyl, or K is (b) (CH₂)ₙCONH(CH₂)ₙNR⁵R^{5a}, where n is 1, 2 or 3, and R⁵ and R^{5a} are as defined above, and 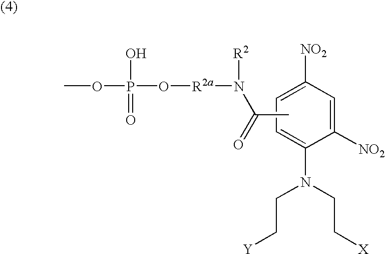
wherein R², R^{2a}, X and Y are as defined in claim 25.

27. A compound as claimed in claim 25, selected from
2-[(2-bromoethyl)-2,4-dinitro-6-({[2-(tetrahydro-2H-pyran-2-yloxy)ethyl]amino}carbonyl)-anilino]ethyl 1-butanesulfonate; and
2-[2-(6-tert-butoxy-8,8-dimethyl-6-oxido-5,7-dioxa-2-aza-6-phosphanon-1-anoyl)(2-chloroethyl)-4,6-dinitroanilino]ethyl methanesulfonate.

28. A compound prepared by a method as claimed in claim 8.
